(19) United States
(12) Patent Application Publication
(10) Pub. No.: US 2014/0206559 A1
(43) Pub. Date: Jul. 24, 2014

(54) ASSAY FOR METASTATIC POTENTIAL OF TUMOR CELLS

(71) Applicants: George M. Whitesides, Newton, MA (US); Ratmir Derda, Edmonton (CA); Renee Fuller, Cambridge, MA (US); Frederique T. Deiss, Cambridge (CA)

(72) Inventors: George M. Whitesides, Newton, MA (US); Ratmir Derda, Edmonton (CA); Renee Fuller, Cambridge, MA (US); Frederique T. Deiss, Cambridge (CA)

(73) Assignee: PRESIDENT AND FELLOWS OF HARVARD COLLEGE, Cambridge, MA (US)

(21) Appl. No.: 14/119,479

(22) PCT Filed: May 23, 2012
(86) PCT No.: PCT/US2012/039058

§ 371 Date: Mar. 31, 2014

Related U.S. Application Data
(60) Provisional application No. 61/488,838, filed on May 23, 2011.

(51) Int. Cl. G01N 33/483

(52) U.S. Cl. CPC G01N 33/4833
USPC 506/9; 435/29; 435/6.12; 506/10; 435/7.92; 435/6.11; 435/7.1; 435/287.8

(57) ABSTRACT

The present invention relates to methods, compositions and kits related to a novel in vitro assay for a high-capacity and high-throughput method for measuring the ability of cancer cells to migrate in a three-dimensional cellular assay. The three-dimensional cellular invasion assay provides a method for determining and quantitating the metastatic potential and invasive capacity of a cancer cell. Other aspects of the invention further relate to the use of the in vitro assay to screen for agents and compounds capable of inhibiting intravasation, and thereby modulating the metastatic potential of cancer cells. The methods, compositions and three-dimensional assay provide a highly sensitive assay system capable of mimicking the in vivo cellular and molecular interactions required for successful completion of intravasation.

### CROSS-REFERENCE TO RELATED APPLICATIONS

This application claims benefit under 35 U.S.C. §119(e) of the U.S. Provisional Application No. 61/488,838 filed May 23, 2011 the content of which is incorporated herein by reference in its entirety.

### GOVERNMENT SUPPORT

The present application was made with Government support under Grant Numbers ES 016665 awarded by National Institutes of Health. The Government of the United States has certain rights in the invention.

### FIELD OF THE INVENTION

The present invention relates generally to methods, devices and assays to measure the invasive potential of cancer and tumor cells, and more particularly to methods, devices and assays to diagnose a subject with metastatic cancer.

### BACKGROUND

One of the greatest problems in the treatment of cancerous tumors is metastasis, i.e., the transmission of cells of a primary tumor to other locations in the patient and the establishment of new tumors at such locations. Metastasis is the primary cause of mortality in cancer; therefore the invasive capacity of cells is a major factor that determines the cancer treatment plan.

The spread of cancer cells from a primary tumor to a site of metastasis formation involves multiple interactions such as the invasion of extracellular matrix, neovascularization, invasion of the blood vessel wall (intravasation), exit from the circulation (extravasation) and establishment of secondary growth. The complexity of the processes involved in metastasis has made it particularly difficult to develop effective treatments to inhibit or prevent the spread of metastatic tumors.

Cancer cells with metastatic potential reach distant sites by disseminating through blood or lymphatic circulation; breaching of the vascular wall is a crucial event in metastasis formation. It is not known at which stage of disease progression that cancer cells acquire the ability to intravasate. However, once established, this pathway appears to remain active as cancer cells can be detected in repeated blood samples of cancer patients.

Moreover, metastasis is difficult to identify and control as metastasis often occurs before a primary tumor is detected and/or diagnosed; the point(s) of metastasis can increase to multiple sites with time and become highly difficult to treat by targeting a single location of metastasis, for example, using radiation or surgery on a specific tumor. Moreover, the metastatic lesions may be in locations which limit the possible dosages of the treatments, e.g., radiation, due to the sensitivity of the surrounding tissue to such treatments. Further, metastatic cells are heterogeneous, and cells which are resistant to conventional therapy tend to emerge.

Histological evidence of invasion usually mandates surgical and/or other aggressive treatments of the tumor. In prostate cancer and breast cancer, which has 217,730 and 207,090 new cases annually in the US, the decision to perform surgical procedures, such as prostatectomy (removal of prostate) and mastectomy (removal of the breast) must be made very carefully. Surgery, though potentially lifesaving, can lead to significant morbidity or mortality. The effect of serious physiological and psychological changes on a patient's life is often severe.

Measuring the invasion of cells isolated from tumors in an in vitro physiological assay can yield results which are complimentary to histological examinations of tumor biopsies. The use of existing invasion assays in a 3D matrix is limited as these typically require complex 3D imaging and time lapse microscopy. Additionally, other high-throughput invasion assays (e.g. Transwell™) have limited capacity to position cells in a 3D environment. Controlled positioning of cells in 3D is possible using microfabricated hydrogels and microfluidics devices, however, these techniques require specialized infrastructure and expertise in microfabrication, and are expensive, require skilled personnel, and can only process a limited number of samples at once. Accordingly, there remains a need for a high-throughput, cost-effective method to efficiently and accurately identify metastatic cells, and cancer cells capable of intravasation, as well as a screen to identify agents and compounds capable of inhibiting metastatic growth.

### SUMMARY

The present invention relates to methods, compositions and kits related to a novel in vitro assay for a high-capacity and high-throughput method for measuring the ability of cancer cells migrate in a three-dimensional cellular assay. The three-dimensional cellular invasion assay, as disclosed herein, provides a method for determining and quantitating the metastatic potential and invasive capacity of a population of cancer cells. In some embodiments, the three-dimensional cellular invasion assay involves seeding a predefined number of cells onto a seed-substrate layer, and monitoring the movement of the cells to one or more, or a series, of stacked receiving substrate layers. In some embodiments, the cells seeded on the seed-substrate layer are cells, such as cancer cells in a population with non-cancer cells, e.g., any cells from a tissue biopsy, such as a tumor tissue biopsy. Other aspects of the invention further relates to the use of the in vitro assay to screen for agents and compounds capable of inhibiting intravasation, and thereby modulating the metastatic potential of cancer cells. The methods, compositions and three-dimensional assay as disclosed herein provides a highly sensitive assay system capable of mimicking the in vivo cellular and molecular interactions required for successful completion of intravasation.

In accordance with the present invention, the inventors have discovered a method to determine the metastatic potential of cells, e.g., cancer and tumor cells using a high-throughput, low cost in vitro assay. In particular, the present invention is related to a quantitative in vitro assay for measuring migration and intravasation of cancer cells. The three-dimensional invasive assay as disclosed herein mimics the physiological phenomenon of metastasis, and traces and measures the quantity the cells once they leave their original positions and invade the ECM/stroma. In some embodiments, a three-dimensional invasive assay as disclosed herein is simple and can be run by robotics (e.g., as an automated system) or by unskilled personnel. In some embodiments, a three-dimensional invasive assay as disclosed herein can be used in high-throughput assays and parallel testing of multiple samples simultaneously.

Such methods, assays and compositions as disclosed herein are useful for identifying the presence of metastatic and highly invasive cancer cells, as well as tools for diagnosis's of a subject having a metastatic cancer, and/or use in a prognostic method to identify the intravasation capacity and aggressiveness of a cancer in a subject, and in an assay to identify agents and compounds which inhibit inhibiting metastatic growth and invasiveness.

In some embodiments, the three-dimensional in vitro assay as disclosed herein measures the invasive capacity of a population of cancer cells by stacking layers of paper, into which a population of cells suspended in a hydrogel have been seeded. Separating each of the layers provides of means assessing the invasion of cancer cells, by looking at each section in the three-dimensional structure, and predicts their potential to metastasize. For example, the three-dimensional in vitro invasive assay as disclosed herein is based upon, in part, the three-dimensional cellular assays disclosed in WO2009/120963, which is incorporated herein in its entirety by reference. In some embodiment, the assay as disclosed herein for measuring intravasation comprises seeding a population of cancer cells in a seed-substrate layer, where the seed-substrate layer is layered with at least one receiving substrate layer, which can be placed atop or below the seed-substrate layer, to form a three-dimensional cellular assay. In some embodiments, the location of the seed-substrate layer can be in any location in the assay, e.g., interdispersed or sandwiched between receiving layers. Such an embodiment is useful to identify direction of the migration of the cells, e.g., to a more oxygenated direction or a less oxygenated direction. For example, where cells are seeded on a seed-substrate located between two receiving substrates, if the cells migrated to the top receiving layer, it would predict the migration of the cells to a regions of more oxygen, whereas if cells migrated to the bottom receiving layer, it would predict cells migrate to regions of less oxygen. This is useful to identify cells which may contribute to a cancers “necrotic core” where the de-differentiation of tumor cells into cancer-like stem cells (which is thought to be related to amount of oxygen). In some embodiments, the amount of cells seeded on the seed-substrate layer is a pre-determined number of cells, for example, at least about 1,000, or at least about 2,000, or at least about 3,000, or at least about 4,000 or at least about 5,000 or about 10,000 cells or more. In some embodiments, the cells seeded on the seed-substrate layer are cells, such as cancer cells in a population of cells comprising non-cancer cells, e.g., any cells from a tissue biopsy, such as a tumor tissue biopsy.

After a pre-determined period of time after seeding and incubation of the three-dimensional cellular assay, the seed-substrate layer is separated from one or more of the receiving substrate layers and the quantity of the cells on one or more receiving layers is measured. The presence or detection of cancer cells that have entered one or more receiving substrate layers of the three-dimensional assay after the pre-defined incubation period is indicative of the invasion of the cancer cell and the metastatic potential of the cancer. The quantity of the cancer cells in the receiving layer can be measured by any method commonly known by one of skill in the art such as imaging or other quantitative methods (e.g., PCR amplification, metabolic profiling, immuno-histological staining, etc). The presence of cancer cells in one or more receiving substrate layers indicates that the cancer cells from the seed-substrate layer are capable of invasion and indicates the cancer cell has a metastatic potential.

Accordingly, aspects of the present invention relate to methods, compositions and kits for detecting and isolating cancer cells with metastatic potential. Cancer cells isolated according to the methods as disclosed herein can be used in a variety of research application, for example, for gene expression assays to study the biological process of metastasis.

The invention further relates to assays for measuring the metastatic potential of such cancer cells and drug screening assays for the identification of agents having anti-metastatic potential. Accordingly, the three-dimensional invasive assay as disclosed herein can be used to screen for agents capable of inhibiting cancer cell intravasation. In some embodiments where such an assay as disclosed herein is used to identify agents with the potential for inhibiting metastasis, a test agent can be included with the population of cancer cells in the seed-substrate layer, or alternatively, in one or more receiving substrate layers, or present in the incubation media surrounding the three-dimensional cellular assay.

In some embodiments, the assay as disclosed herein is also useful in methods to detect a phenotypic change effected by genetic manipulation of cancer cells that results in changes in metastatic potential. Additionally, in other embodiments, the assay can be used in conjunction with new gene discovery methods to test the role of newly discovered genes in control of metastasis.

Accordingly, disclosed herein are novel methods for the detection and isolation of cancer cells with metastatic potential from blood, ascites and tumor tissue derived from subjects with metastatic cancer.

Other aspects of the present invention relate to novel compositions for detection and isolation of cancer cells and for use as to identify subjects having metastatic cancers. The methods and compositions of the invention may also be used in assays designed for measuring the metastatic potential of isolated cancer cells and for identification of agents having anti-metastatic potential.

In addition, the invention relates to inhibiting the metastatic potential of cancer cells by modulating the activity using inhibitors of actin-based mobility on the surface of the seed-substrate and/or receiving substrate. The present invention is based on the discovery that metastatic cancer cells migrate through a three-dimensional cellular assay at an increased rate and increased percentage as compared to non-metastatic cancer cells.

One aspect of the present invention relates to a method for assessing the metastatic potential of a population of cancer cells, the method comprising: (a) contacting a biological sample comprising a population of cancer cells and a hydrogel at a defined region of seed-substrate, wherein the seed-substrate is a porous, hydrophilic substrate of a three-dimensional cellular assay, wherein the seed-substrate is in contact with at least one receiving-substrate to form a three-dimensional cellular assay, wherein the receiving-substrate is a porous, hydrophilic substrate comprising a hydrogel at defined regions, (b) separating the seed-substrate and the at least one receiving-substrates of the three-dimensional cellular assay after incubation of the three-dimensional cellular assay for a predefined period of time; (c) measuring the quantity of cancer cells on the at least one receiving-substrate, where the amount of cancer cells on the receiving-substrate is indicative the metastatic potential of the population of cancer cells.

In some embodiments, a seed-substrate is in contact with at least one lower-receiving substrate, or a seed-substrate is in contact with at least one upper-receiving substrate, or a seed-substrate is in contact with at least one upper-receiving substrate and at least one lower substrate.

In some embodiments, a three-dimensional cellular assay as disclosed herein comprises a plurality of receiving substrates, wherein the each receiving substrate is layered on at least one other receiving substrate [e.g., a multi-layered receiving-substrate], and can comprise between about 1 and 50 receiving substrates, or between about 50 and 100 receiving substrates, or any number in between.

In some embodiments, at least one receiving substrate can comprise extracellular matrix (ECM), or alternatively at least one additional cell type in the hydrogel, for example, where an additional cell can be selected from the group of endothelial cells, stromal cells, fibroblast cells, or any combination thereof. In some embodiments, the receiving substrate can comprise other extracellular matrices, for example, MatriGel, collagen, laminin, gelatin, fibronectin, and other EMCs known to one of ordinary skill in the art.

In some embodiments, a seed-substrate and the at least one receiving-substrate can comprise a matrix with a pore-diameter to allow invasion of metastatic cells, and can be paper, or cellulose-based substrates and scaffolds. In some embodiments, the receiving substrates can be polymeric, nitrocellulose, cellulose acetate etc. In some embodiments, the substrates can be plastic or any substrate with pre-defined or irregular pore sizes, including ceramic substrate with a pre-defined pore size.

In some embodiments, one can compare the quantity of cancer cells on the seed-substrate with the quantity of cancer cells on at least one receiving-substrate at a predefined timepoint. In some embodiments, a biological sample comprises a predefined number of cancer or tumor cells, and one can compare the predefined number of cancer cells in a biological sample with the measured quantity of cancer cells on the receiving substrate at a predefined timepoint. In other embodiments, when a high percentage of cancer cells remain on the seed-substrate, as compared to the percentage of cells that migrate onto the receiving substrate, is indicative of a population of cancer cells with low metastatic potential.

In some embodiments, a biological sample comprises a portion of a cancer biopsy sample, or can comprise a cell suspension of cancer cells, or alternatively, can comprise cancer cells which were obtained from a tumor biopsy obtained from a subject. In some embodiments, one can label the cancer cells in the biological sample prior to contact with the seed-substrate. In other embodiments the cancer cells can be labeled after placed in the seed-substrate, either as an assembled three-dimensional cellular assay or after the seed-substrate is separated from at least one receiving substrate. In some embodiments, the cancer cells are fixed in the three-dimensional cellular assay prior to, or after separation of the seed-substrate with the at least one receiving substrate.

In some embodiments, the quantity of the cancer cells is measured by image analysis, for example, cell proliferation assays, staining by substrates processed by live cells (e.g., metabolic activity stains), fluorescent labeling assays, immunostaining for specific marker (e.g. cytokeratin) and staining of the specific cellular compartments (e.g. total DNA, mitochondrial activity, membrane, etc.). In some embodiments, a quantity of the cancer cells can be measured after the cancer cells are removed from the seed-substrate or at least one receiving substrate, for example, the cells can isolated from the seed-substrate and/or the receiving substrate and be counted by one of ordinary skill in the art using standard cytometry assays (e.g. heamocytometer, automated cell counter, flow cytometer and the like).

In some embodiments, the quantity of the cancer cells is measured by quantifying the level of expression of specific genes or proteins.

Another aspect of the present invention relates to a system, for example, a computerized system for automated processing of the methods as disclosed herein. For example, such a system as disclosed herein is useful for a high-capacity 3D in vitro invasive assay, where the invasive potential of many biological samples comprising cancer cells can be processed simultaneously. For example, in some embodiments a system can comprise an assay module, a determination module, a storage module, an output or display module, which are connected to a processor (See FIG. 3). In some embodiments, the processor is connected to a database, and optionally a network (see FIG. 5).

As exemplified by FIG. 3, one embodiment of a system for assessing the metastatic potential of a population of cancer cells in a biological sample, comprises an assay module, a determination module, a storage module, a comparison module, and a display module, which are all connected to a processor. The assay module is version of robotic plate handler, e.g., an automated plate handler, which can seed cancer cells from a biological samples sources (e.g., from 12-well, 24-well, 48-well, 96-well, 386-well microwell plates) onto the predefined regions of hydrogel onto a seed-substrate. In some embodiments, the assay module positions the seed-substrate (with the seed cancer cells) in a location (e.g., an incubation chamber) in the assay module for incubation of the seed-substrate prior to stacking with at least one receiving substrate.

After incubation of the seed-substrate, the assay module positions the seed-substrate to stack the seed substrate with at least one or more receiving substrates and positions the 3D invasive assay in a location (e.g., an incubation chamber in the assay module) for a predetermined time. After incubation for a pre-determined period time, a robotic arm in the assay module separate the seed-substrate(s) from at least one receiving substrate and presents the separated substrates to the determination module.

The assay module is connected to a processor which can have a user interface for programming the robotics of the assay module by the user (e.g., the time of incubation of the seed-substrate and the times of incubation of the 3D-invasion assay, the number of receiving substrates in the 3D-invasive assay and the like).

The assay module can present the separated substrates (e.g., receiving substrates and seed-substrates) to the determination module, which is a reading device or data gathering device to measure the amount of cells on the receiving substrate and optionally on the seed-substrates. The determination module can measure the amount of cells using any method as disclosed herein. In some embodiments, the determination module is an image scanner. The determination module gathers data and outputs the data (e.g., receiving substrate data and seed-substrate data) to a processor, which can be stored on the connected storage module.

In some embodiments, the processor can be connected to a comparison module, which comprises software to compares the output data (e.g., receiving substrate data and seed-substrate data) of the test cancer cell line with at least one reference cancer cell line and generates report data. The comparison module comprises computer readable media with instructions for comparisons, for example, exemplary processing instructions are shown in FIG. 4, however other comparisons as encompassed in the present invention as disclosed herein. In some embodiments a comparison module provides a report data, which is reported on a display module or other output report.

Also disclosed herein is a method to identify metastatic cancer cells as compared to non-metastatic cancer cells. Using the three-dimensional cellular assay as disclosed herein, cells which migrate through the three-dimensional matrixes at a faster rate and/or a higher proportion of cells can be distinguished from non-migratory non-metastatic cells.

In further experiments as disclosed herein, the inventors have validated the presence of a population of metastatic cancer cells identified by the assay by comparing them with a non-metastatic cancer cells population.

Accordingly, in one embodiment the present invention is related to methods to identify a population of metastatic cells in a population of cells. Another embodiment is related to a method of identifying a cell population which is highly invasive, and is predictive of high metastatic potential of a cancer.

Another embodiment of the present invention is related to methods to identify and enrich for a populations of metastatic cancer cells, and uses of enriched population of metastatic cancer cells to study cancer metastasizes and the like.

### BRIEF DESCRIPTION OF FIGURES

FIG. 1 shows images of the upper receiving layer, the seed layer and lower layer of non-invasive (left) and invasive cells (right) of a 96-zone seed layer after the completion of invasion assay. Different rows were stimulated in the presence and absence of different agents (serum, TGFβ) and presence or absence of inhibitors (CytoclasinD, SB-431542). Each combination was done in 8-duplicate wells per cell line. FIG. 1 shows that non-invasive cells (left) did not invade the upper receiving layer or the lower receiving layer, whereas the invasive cells invaded the upper receiving layer.

FIGS. 2A-2B show histograms of % invasion of cells in the upper receiving layer; results form 5 cell lines are presented. FIG. 2A shows invasion of cells in paper-Matrigel layers after cultured for 48 hours in normal serum containing medium. FIG. 2B shows invasion of cells in paper-Matrigel layers after cultured for 24 hours in serum-free medium followed by culturing for 48 hours with normal serum during the invasion assay.

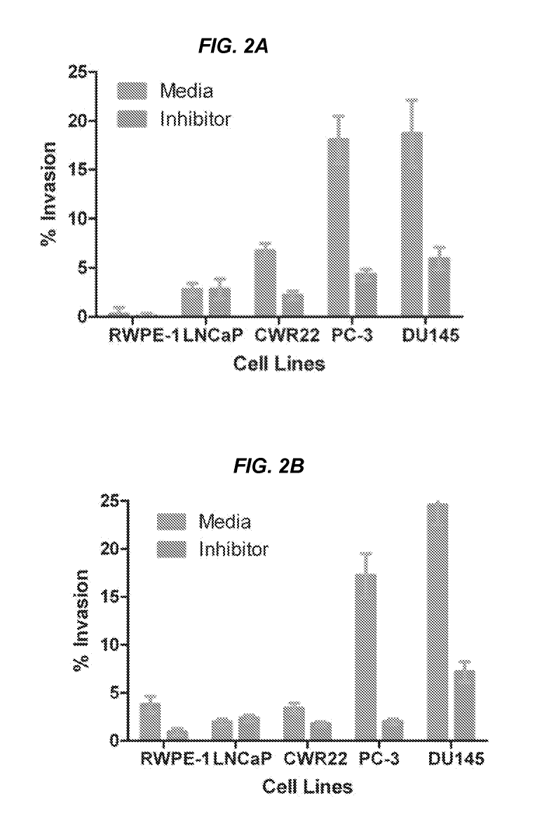

FIG. 3 is a block diagram showing an example of an embodiment of a system for assessing the metastatic potential of a population of cancer cells in a biological sample, the system comprising an assay module, a determination module, a storage module, a comparison module, and a display module, which are all connected to a processor. The assay module is an automated robotic assay handler, similar to a robotic plate handler, which seeds the cancer cells from a cell sample source (e.g., from a microwell plate) onto the predefined regions on a seed-substrate. In some embodiments, the assay module positions the seed substrate in a location (e.g., an incubation chamber) in the assay module for incubation of the seed-substrate prior to stacking with at least one receiving substrate. After incubation of the seed-substrate, the assay module positions the seed-substrate to stack the seed substrate with at least one or more receiving substrates and positions the 3D invasive assay in a location (e.g., an incubation chamber in the assay module) for a predetermined time. After incubation for a pre-determined period time, a robotic arm in the assay module separate the seed-substrate(s) from at least one receiving substrate and presents the separated substrates to the determination module, which is a reading device or data gathering device to measure the amount of cells on the receiving substrate and optionally on the seed-substrates. The assay module is connected to a processor which can have a user interface for programming the robotics of the assay module by the user. The amount of cells on each separated substrate (e.g., receiving substrates and seed-substrates) can be measured on a determination module, and the output data (e.g., receiving substrate data and seed-substrate data) can be stored on the storage module. The processor can be connected to a comparison module, which comprises software to compares the output data (e.g., receiving substrate data and seed-substrate data) of the test cancer cell line with a reference cancer cell line and generates report data. The comparison module provides a report data, which is reported on a display module or other output report.

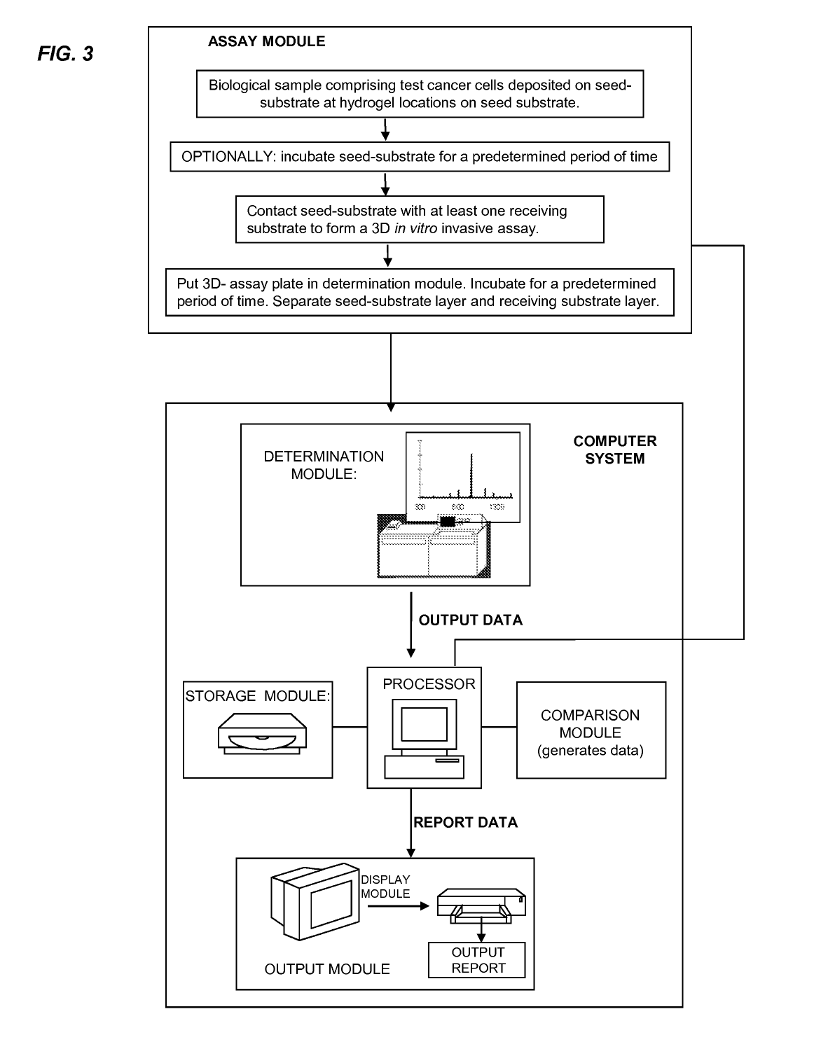

FIG. 4 is a block diagram showing exemplary instructions on a computer readable medium for assessing the metastatic potential of a population of cancer cells in a biological sample and whether a subject from whom the cancer cell population was obtained has, or is at risk of having a metastatic cancer or cancer with invasive capacity.

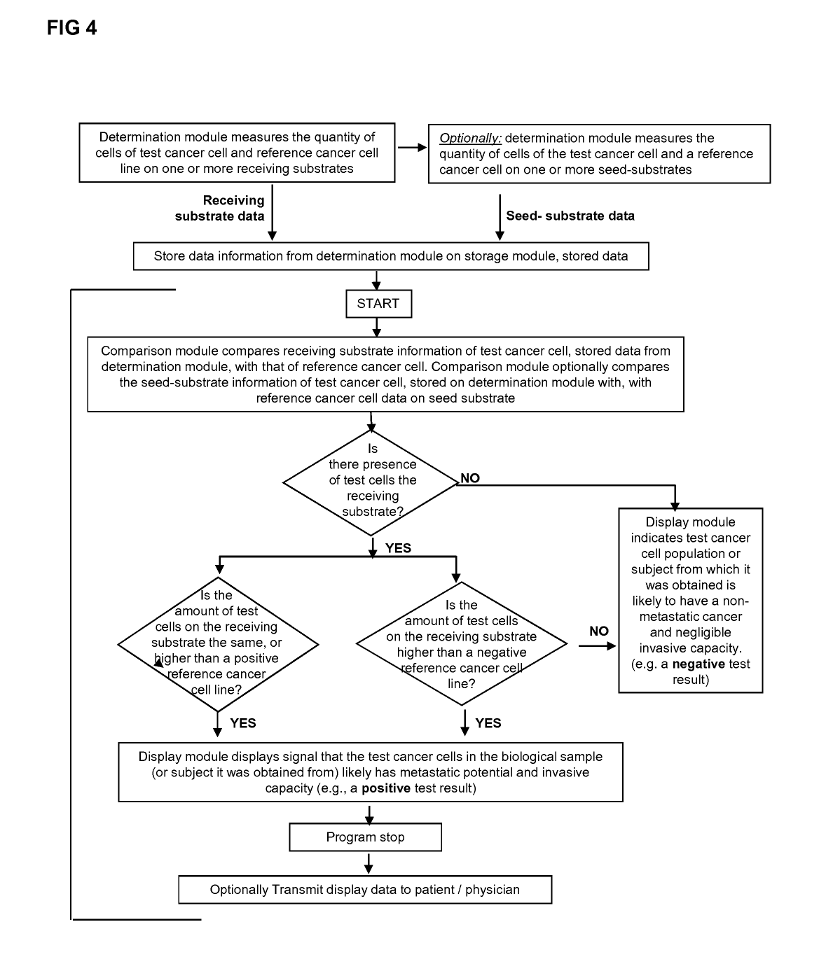

FIG. 5 shows a simplified block diagram of an embodiment of the present invention which enables the data from the processor to be configured to be processed by a computer system at any location and accessible through a user interface, where the output data (e.g., receiving substrate data and seed-substrate data) or report data generated from the comparison module is stored in a database.

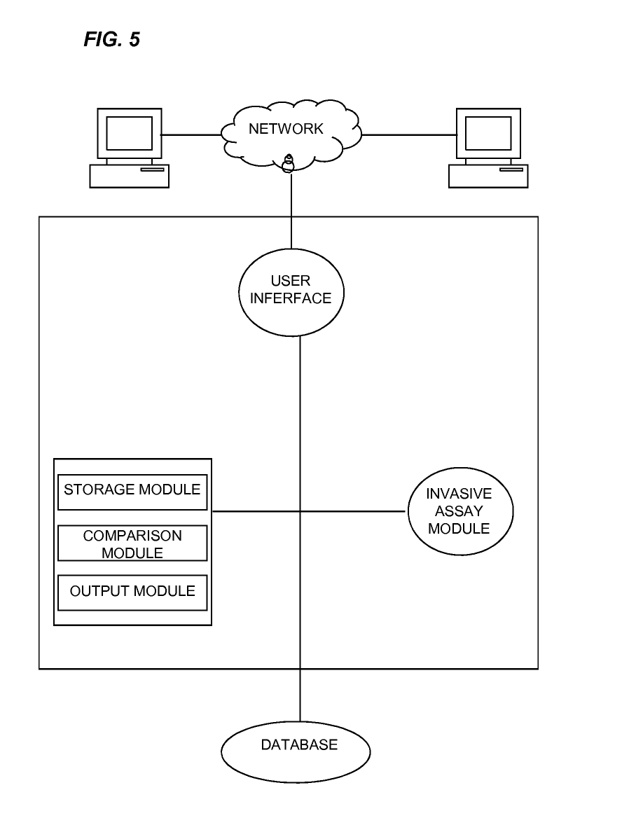

FIG. 6 shows a schematic and images of the folded layer used to culture primary breast cancer cells from a human patient. Cells were isolated from 2 mm core biopsy using collagenase and plated in Matrigel at 3×10⁶ cells/mL in the indicated zones. The structure was folded, cultured for 5 days, unfolded and stained with calcein. Black color is proportional to calcein fluorescence. Black ellipses on the top and the bottom of the raw image are reference markers made by ink (they are NOT cells). The image on the bottom describes radial distribution of calcein intensity in the stack. Black color is proportional to the intensity of green fluorescence.

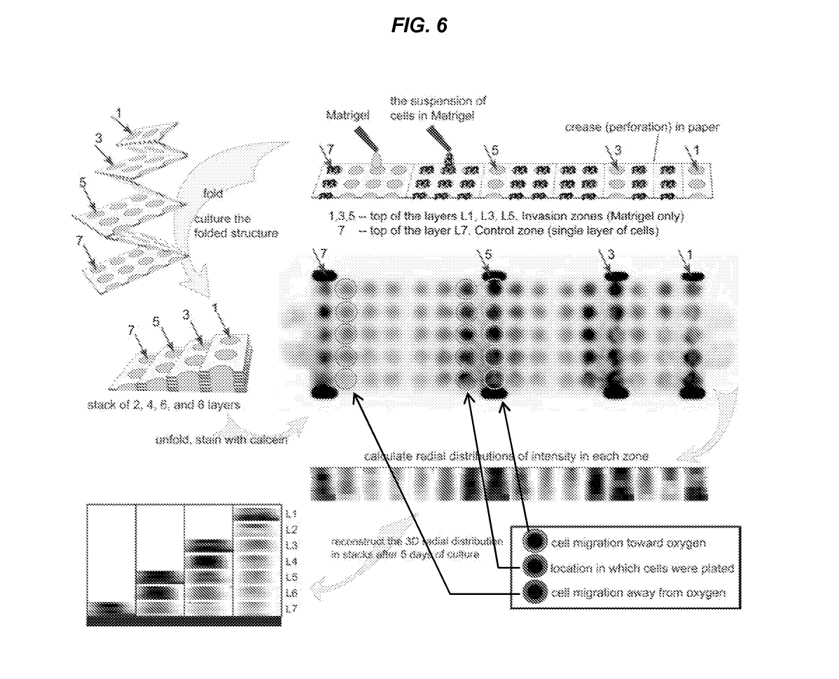

### DETAILED DESCRIPTION OF THE INVENTION

As disclosed herein, one aspect of the present invention relates to methods for assessing the metastatic potential of a population of cancer cells by measuring the quantity of cancer cells which have migrated through layers in a three-dimensional cellular assay after a pre-determined period of time.

The present invention is based upon the discovery that cancer cells which have a high intravasation ability can migrate from the seed layer of a three-dimensional cellular assay to receiving layers; measuring the quantity of cells which are present on a receiving substrate layer of a three-dimensional cellular assay, after a pre-defined period of time is a measure of the metastatic potential of cells. Accordingly, the present invention is related to a quantitative in vitro assay for measuring the migration and intravasation of cancer cells and provides a method for assessing and quantitating the metastatic potential of a cancer cell. Accordingly, one aspect of the present invention relates to a three-dimensional in vitro assay for measuring the invasive capacity of a population of cancer cells, where the three-dimensional cellular assay comprises stacking a cell-containing layer, herein referred to as a “seed-substrate layer” on a receiving substrate layer. The receiving layers can be separated from the seed-substrate layer, and the number of cells in each layer can be determined; this assesses the invasion of the cancer cells and predicts their metastatic potential. For example, the three-dimensional in vitro invasive assay as disclosed herein is based upon, in part, the three-dimensional cellular assays disclosed in WO2009/120963, which is incorporated herein in its entirety by reference. In some embodiment, the assay as disclosed herein for measuring intravasation comprises seeding a population of cancer cells in a seed-substrate layer, where the seed-substrate layer is layered with at least one receiving substrate layer to form a three-dimensional cellular assay. After a pre-determined period of time after seeding and incubation of the three-dimensional cellular assay, the seed-substrate layer is separated from one or more of the receiving substrate layers and the quantity of the cells on one or more receiving layers is measured. Stated another way, in some embodiments, the assay comprises (1) seeding cells into a seed-substrate layer, (2) incubating the seed-substrate layer for a pre-defined time period to allow ECM to solidify and the cells to grow in the substrate, (3) stacking the seed-substrate layer with receiving substrate layers to make a 3D structure of the seed-substrate layer and multiple receiving layers (in any configuration), (4) incubating the 3D structure for a predefined time period, (5) destacking the receiving substrate layers from the seed substrate and evaluating one or more layers for the presence of the cells. In some embodiments, one can measure the cells on one or more receiving layer and also on the seed-substrate layer. In some embodiments, the time period for step 2 enables the cells to remain stable and not migrating at the time of the assay construction (e.g., layering the seed-substrate with the receiving substrate layers). In some embodiments, the time period for step (4) is sufficient to evaluate movement of the cells between the seed-substrate layer and one or more of the receiving layers.

The presence or detection of cancer cells that have entered one or more receiving substrate layers of the three-dimensional assay after the pre-defined incubation period is indicative of the invasion of the cancer cell and the metastatic potential of the cancer. The quantity of the cancer cells in the receiving layer can be measured by any method commonly known by one of skill in the art, e.g., by imaging or other quantitative methods, e.g., PCR amplification etc. The presence of cancer cells in one or more receiving substrate layers indicates that the cancer cells from the seed-substrate layer are capable of invasion and indicates the cancer cell has a metastatic potential.

Other aspects of the invention further relates to the use of the in vitro assay to screen for agents capable of inhibiting intravasation, and thereby modulating the metastatic potential of cancer cells. The methods, compositions and three-dimensional assay as disclosed herein provides a highly sensitive assay system capable of mimicking the in vivo cellular and molecular interactions required for successful completion of intravasation.

Such methods, assays and compositions as disclosed herein are useful for identifying the presence of metastatic and highly invasive cancer cells, as well as tools for diagnosis's of a subject having a metastatic cancer, and/or use in a prognostic method to identify the intravasation capacity and aggressiveness of a cancer in a subject, and in an assay to identify agents and compounds which inhibit inhibiting metastatic growth and invasiveness.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term ‘malignancy’ and ‘cancer’ are used interchangeably herein, and refers to diseases that are characterized by uncontrolled, abnormal growth of cells. Cancer cells can spread locally or through the bloodstream and lymphatic system to other parts of the body. The term is also intended to include any disease of an organ or tissue in mammals characterized by poorly-controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer.

As used herein, “metastasis” refers to the ability of cells of a cancer (e.g. a primary tumor, or a metastasis tumor) to be transmitted to other locations in the subject and to establish new tumors at such locations. An agent that “inhibits” cancer metastasis may function at any of a variety of steps in metastatic progression. For example, it may result in the delayed appearance of secondary tumors, slowed development of primary or secondary tumors, decreased occurrence of secondary tumors, slowed or decreased severity of secondary effects of disease, arrested tumor growth and regression of tumors, among others. In the extreme, complete inhibition is referred to herein as prevention (e.g., virtually complete inhibition, no metastasis if it had not occurred, no further metastasis if there had already been metastasis of a cancer, or virtually complete inhibition of the growth of a primary tumor caused by re-seeding of the tumor by a metastasized cell.

A “metastatic” cell, as used herein, refers to a cell that has a potential for metastasis and, when used in a method of the invention, is able to seed a tumor or a cell colony of interest. A “highly metastatic” cell, as used herein, refers to a cell that has a high potential for metastasis; e.g., cells from a cell line such as, but not limited to LM2, MDA-MB-231, PC-3, DU-145, Lewis Lung carcinoma, as described herein, can be considered to be highly metastatic cells. Metastatic cells can be generated in a variety of ways, which are discussed further below.

A “tumorigenic cell,” as used herein, is a cell that, when introduced into a suitable site in a subject, can form a tumor. The cell may be non-metastatic or metastatic. A variety of types of tumorigenic and/or metastatic cells can be used in a method of the invention, including cells from metastatic epithelial cancers, carcinomas, melanoma, leukemia, etc. The tumor cells may be, e.g., from cancers of breast, lung, colon, bladder, prostate, liver, gastrointestinal tract, endometrium, tracheal-bronchial tract, pancreas, liver, uterus, ovary, nasopharynges, prostate, bone or bone marrow, brain, skin or other suitable tissues or organs. In a preferred embodiment, the cancer cells are of human origin.

The term “tumor” or “tumor cell” are used interchangeably herein, and refers to the tissue mass or tissue type of cell that is undergoing abnormal proliferation.

A “cancer cell” refers to a cancerous, pre-cancerous or transformed cell, either in vivo, ex vivo, and in in vitro tissue culture, that has spontaneous or induced phenotypic changes that do not necessarily involve the uptake of new genetic material. Although transformation can arise from infection with a transforming virus and incorporation of new genomic nucleic acid, or from the uptake of exogenous nucleic acid, it can also arise from spontaneous mutations within the genome that follow exposure to a carcinogen or other DNA-altering substance. Transformation/cancer is associated with, e.g., morphological changes, immortalization of cells, aberrant growth control, foci formation, anchorage dependence, proliferation, malignancy, contact inhibition and density limitation of growth, growth factor or serum dependence, tumor specific markers levels, invasiveness, tumor growth or suppression in suitable animal hosts such as nude mice, and the like, in vitro, in vivo, and ex vivo (see Example VII) (see also Freshney, Culture of Animal Cells: A Manual of Basic Technique (3rd ed. 1994)).

A “sarcoma” refers to a type of cancer cell that is derived from connective tissue, e.g., bone (osteosarcoma) cartilage (chondrosarcoma), muscle (rhabdomyosarcoma or rhabdosarcoma), fat cells (liposarcoma), lymphoid tissue (lymphosarcoma), collagen-producing fibroblasts (fibrosarcoma). Sarcomas may be induced by infection with certain viruses, e.g., Kaposi's sarcoma, Rous sarcoma virus, etc.

The term “tissue” is intended to include intact cells, blood, blood preparations such as plasma and serum, bones, joints, muscles, smooth muscles, and organs.

The term “disease” or “disorder” is used interchangeably herein, and refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, inderdisposion, affection.

As used herein the terms “patient”, “subject” and “individual” are used interchangeably herein, and each refer to any living organism in which a cancer or a proliferative disorder can occur and where assessing the cancer for invasive potential and identifying a metastatic cancer is beneficial. A subject is also any mammal where identifying a metastatic cancer is beneficial and where treatment including prophylactic treatment can be provided. The term “subject” as used herein refers to human and non-human mammals. The term includes, but is not limited to, humans, non-human animals, for example non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult and newborn subjects, as well as fetuses, whether male or female, are intended to be covered. The term “subject” also includes living organisms susceptible to conditions or disease states as generally disclosed, but not limited to, throughout this specification. Examples of subjects include humans, dogs, cats, cows, goats, and mice, including transgenic species The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. The appropriate cell culture media, for a particular cell type, is known to those skilled in the art.

As used herein, the term “biological sample” also refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from a subject, but the term can also refer to non-cellular biological material, such as non-cellular fractions of blood, saliva, or urine, that can be used to measure protein phosphorylation levels. As used herein, a “biological sample” or “tissue sample” refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, a biological sample is from a resection, bronchoscopic biopsy, or core needle biopsy of a primary, secondary or metastatic tumor, or a cellblock from pleural fluid. In addition, fine needle aspirate biological samples are also useful. In some embodiments, a biological sample is primary ascite cells. Samples can be fresh, frozen, fixed or optionally paraffin-embedded, frozen or subjected to other tissue preservation methods, including for example methods to preserve the phosphorylation status of polypeptides in the biological sample. A biological sample can also mean a sample of biological tissue or fluid that comprises protein or cells. Such samples include, but are not limited to, tissue isolated from subjects or animals. Biological samples may also include sections of tissues such as biopsy and autopsy samples, frozen sections taken for histological purposes, blood, plasma, serum, sputum, stool, tears, mucus, hair, and skin. Biological samples also include explants and primary and/or transformed cell cultures derived from patient tissues. A biological sample may be provided by removing a sample of cells from subject, but can also be accomplished by using previously isolated cells (e.g., isolated by another person, at another time, and/or for another purpose), or by performing the methods of the invention in vivo. Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. The biological sample can be obtained by removing a sample of cells from a subject, but can also be accomplished by using previously isolated cells (e.g. isolated by another person), or by performing the methods of the invention in vivo. Such samples include, but are not limited to, whole blood, cultured cells, primary cell preparations, sputum, amniotic fluid, tissue or fine needle biopsy samples, peritoneal fluid, and pleural fluid, among others. In some embodiments a biological sample is taken from a human patient, and in alternative embodiments the biological sample is taken from any mammal, such as rodents, animal models of diseases, commercial animals, companion animals, dogs, cats, sheep, cattle, and pigs, etc. The biological sample can be pretreated as necessary for storage or preservation, by dilution in an appropriate buffer solution or concentrated, if desired. Any of a number of standard aqueous buffer solutions, employing one of a variety of buffers, such as phosphate, Tris, or the like, at physiological pH can be used. The biological sample can in certain circumstances be stored for use prior to use in the assay as disclosed herein. Such storage can be at +4 C or frozen, for example at −20 C or −80 C, provided suitable cryopreservation agents are used to maintain cell viability once the cells are thawed.

The term “drug screening” as used herein refers to the use of cells and tissues in the laboratory to identify drugs with a specific function. In some embodiments, the present invention provides drug screening methods of to identify compounds or drugs which inhibit the invasiveness of a cancer cell. In alternative embodiments, the present invention provides drug screening on cancer cells to identify compounds or drugs useful as therapies for diseases or illnesses (e.g. human diseases or illnesses), e.g., for the treatment of metastatic cancer.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found on the surface or in the interior of a cell including, but not limited to, proteins (peptides and polypeptides), lipids, polysaccharides, nucleic acids, and metabolites such as steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

The term ‘lineages” as used herein refers to a term to describe cells with a common ancestry, for example cells that are derived from the same ovarian cancer stem cell.

As used herein, the term “clonal cell line” refers to a cell lineage that can be maintained in culture and has the potential to propagate indefinitely. A clonal cell line can be a stem cell line or be derived from a stem cell, and where the clonal cell line is used in the context of clonal cell line comprising stem cells, the term refers to stem cells which have been cultured under in vitro conditions that allow proliferation without differentiation for months to years. Such clonal stem cell lines can have the potential to differentiate along several lineages of the cells from the original stem cell.

As used herein, the term “treating” includes preventing the progression and/or reducing or reversing at least one adverse effect or symptom of a condition, disease or disorder associated with inappropriate proliferation, for example cancer. The term “treating” also includes reducing or alleviating at least one adverse effect or symptom of a condition, disease or disorder associated with inappropriate proliferation, for example cancer. As used herein, the term treating is used to refer to the reduction of a symptom and/or a biochemical marker of in appropriate proliferation, for example a reduction in at least one biochemical marker of cancer by at least 10%. For example but are not limited to, a reduction in a biochemical marker of cancer, for example a reduction in, as an illustrative example only, at least one of the following biomarkers; CD44, telomerase, TGF-α, TGF-β, erbB-2, erbB-3, MUC1, MUC2, CK20, PSA, CA125, FOBT, by 10%, or a reduction in the rate of proliferation of the cancer cells by 10%, would be considered effective treatments by the methods as disclosed herein. As alternative examples, a reduction in a symptom of cancer, for example, a slowing of the rate of growth of the cancer by 10% or a cessation of the increase in tumor size, or a reduction in the size of a tumor by 10% or a reduction in the tumor spread (i.e. tumor metastasis) by 10% would also be considered as affective treatments by the methods as disclosed herein.

The term “effective amount” as used herein refers to the amount of at least one agent of pharmaceutical composition to reduce or stop at least one symptom of the abnormal proliferation, for example a symptom of a cancer or malignancy. For example, an effective amount using the methods as disclosed herein would be considered as the amount sufficient to reduce a symptom of the abnormal proliferation, for example at least one symptom of a cancer or malignancy by at least 10%. An effective amount as used herein would also include an amount sufficient to prevent or delay the development of a symptom of the disease, alter the course of a symptom disease (for example but not limited to, slow the progression of a symptom of the disease), or reverse a symptom of the disease.

As used herein, the terms “administering,” and “introducing” are used interchangeably, and refer to the placement of the agents as disclosed herein into a subject by a method or route which results in at least partial localization of the agents at a desired site. The compounds of the present invention can be administered by any appropriate route which results in an effective treatment in the subject.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of cardiovascular stem cells and/or their progeny and/or compound and/or other material other than directly into the central nervous system, such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, or be biologically inert.

The term “agent” refers to any entity which is normally not present or not present at the levels being administered in the cell. Agent may be selected from a group comprising, for example chemicals; small molecules; nucleic acid sequences; nucleic acid analogues; proteins; peptides; peptidomimetics, aptamers; antibodies; or fragments thereof. A nucleic acid sequence may be RNA or DNA, and may be single or double stranded, and can be selected from a group comprising; nucleic acid encoding a protein of interest, oligonucleotides, antisense oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), short-temporal RNAi (stRNA), dsRNA antisense oligonucleotides etc. A chemical entity or biological product is preferably, but not necessarily a low molecular weight compound, but may also be a larger compound, or any organic or inorganic molecule, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds. Agents can be, without limitation an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. The agent may be applied to the media, where it contacts the ovarian cell and induces its effects. Alternatively, the agent may be intracellular within the cell as a result of introduction of the nucleic acid sequence into the cell and its transcription resulting in the production of the nucleic acid and/or protein agent within an ovarian cancer cell.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “enriching” is used synonymously with “isolating” cells, means that the yield (fraction) of cells of one type is increased over the fraction of other types of cells as compared to the starting or initial cell population. Preferably, enriching refers to increasing the percentage by about 10%, by about 20%, by about 30%, by about 40%, by about 50% or greater than 50% of one type of cell in a population of cells as compared to the starting population of cells.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, refer to a population of cells that contain fewer than about 20%, more preferably fewer than about 15%, 10%, 8%, 7%, most preferably fewer than about 5%, 4%, 3%, 2%, 1%, or less than 1%, of cells that are not cardiovascular stem cells or cardiovascular stem cell progeny of the invention.

As used herein, “protein” is a polymer consisting of any of the 20, essential, amino acids as well as amino acids that are post-translationally modified. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The term “inhibition” or “inhibit” when referring to the gene expression and/or activity of a protein refers to a reduction or prevention in the level of its function or a reduction of its gene expression product.

The terms “decrease”, “reduced”, “reduction”, or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” is used interchangeably herein with “program” and refers to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

The term “proteomics” may refer to the study of the expression, structure, and function of proteins within cells, including the way they work and interact with each other, providing different information than genomic analysis of gene expression.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean ±1%. The present invention is further explained in detail by the following, including the Examples, but the scope of the invention should not be limited thereto.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### High Throughput Three-Dimensional Invasive Assay

In some embodiments, a three-dimensional in vitro invasive assay as disclosed herein (also referred interchangeably herein as a “metastatic assay” or “migration assay”) is based upon, in part, the three-dimensional cellular assays disclosed in International Application WO2009/120963, which is incorporated herein in its entirety by reference.

In some embodiment, the assay as disclosed herein for measuring intravasation comprises seeding a population of cancer cells on a seed-substrate layer, where the seed-substrate layer is layered with at least one receiving substrate layer to form a three-dimensional cellular assay.

In some embodiments, a substrate, e.g., a seed-substrate or receiving substrate comprises at least one porous region which comprises a hydrogel or other similar growth media embedded within the porous regions, and where the porous regions are surrounded by liquid impervious boundary, as disclosed in International Application WO2009/120963. For embodiments where it is desirable to measure the invasiveness of many different cancer cell populations at the same time, e.g., for a high-capacity and/or high-throughput invasive assay, a substrate can comprise a plurality of porous regions comprising hydrogels and/or other growth media, for assessing the invasive potential of a plurality of different cancer cell populations. In some embodiments, a substrate adapted for a high-capacity invasive assay comprises at least about 12, or at least about 24, or at least about 48, or at least about 96 or at least about 384, or more porous regions. In some embodiments, a substrate adapted for high-capacity invasive assay comprises an array of about 1, 2, 3, 4, 5, 10, 25, 50, 100, 150, 200, 250, 300, 400, 500, 1,000 or more porous regions, each bound by a liquid impervious boundary. For example, a seed-substrate that comprises a plurality of different cancer populations can be an array of different types of cancer cells, and/or cancer cells of the same type from a number of different subjects, as well as reference control cells (e.g., negative reference control cells which are non-migratory cells, and/or positive reference control cells which are known to be highly migratory) and reporter cell lines. Thus, the present invention allows for a co-culture of multiple biological samples in the same 3D-invasive assay, where the migratory ability of the cancer cells in each biological sample can be compared to one another after the assay is performed and the seed-substrate is separated from one or more receiving substrates.

A biological sample comprising a population of cancer cells is seeded onto the seed-substrate layer at locations of the substrate which comprise a growth media or hydrogel, for example, Matrigel, collagen, iontrophic hydrogel and the like, although other growth medias can be used. Exemplary hydrogels which can be used are disclosed in International Application WO2009/120963. A seed-substrate can comprise the same population of cells at different hydrogel locations, e.g., for duplicate tests of the same cell population, or a variety of different cell populations in different hydrogel locations in the same seed substrate.

In some embodiments, the three-dimensional in vitro invasive assay comprises a seed-substrate which is layered with at least one receiving layer. In some embodiments, the receiving layer is an upper receiving layer. In alternative embodiments, the receiving layer is a lower receiving layer. In some embodiments, a three-dimensional in vitro invasive assay comprises a seed-substrate which is layered with at least one upper receiving layer, and at least one lower receiving layer so that the seed substrate is sandwiched between the upper and lower receiving layers. In some embodiments, a three-dimensional in vitro invasive assay comprises at least one seed-substrate which is layered with a plurality of receiving layers, which can be any combination of upper and/or lower receiving layers. For example but by no way a limitation, a three-dimensional in vitro invasive assay as disclosed herein can comprise at least 2, or at least 3, or at least 4, or at least 5, or at least about 6, or at least about 7, or at least about 8, or at least about 9, or at least about 10, or at least about 12, or at least about 15, or at least about 18, or at least about 20, or more than 20 receiving substrate layers, which can be any combination of upper and/or lower receiving substrate layers. In some embodiments, a three-dimensional in vitro invasive assay as disclosed herein can comprise between about 20-50 receiving substrate layers, or between about 50-100 receiving substrate layers, or any integer between 20-50 receiving layers. As an exemplary example, a 3D-invasion assay as disclosed herein can comprise at least about 5 upper receiving substrates layers, a least one seed-substrate layer (or a plurality of seed-substrate layers) and at least about 2 lower substrate layers. Stated another way, in some embodiments a three-dimensional in vitro invasive assay can comprise, for example, the following configuration; a plurality of upper receiving substrate layers (e.g., a stack of upper receiving substrate layers), where the inner-most upper receiving substrate layer is in contact with at least one seed-substrate layer (although in some embodiments there can also be a plurality of stacked seed-substrate layers), and where the seed-substrate layer can optionally be in contact with at least one lower receiving layers, or a plurality of lower receiving layers, (e.g., a stack of lower receiving substrate layers).

Similarly, in some embodiments, a three-dimensional in vitro invasive assay can comprise a plurality of seed-substrate layers which is layered with at least one receiving layer, for example, at least 2, or at least 3, or at least 4, or at least 5, or at least about 6, or at least about 7, or at least about 8, or at least about 9, or at least about 10, or at least about 12, or at least about 15, or at least about 18, or at least about 20, or more than 20 seed-substrate layers.

In some embodiments, a receiving substrate layer which is stacked on the seed-substrate layer can also comprise the same or different growth media or hydrogel, for example, Matrigel, at the same locations. In some embodiments the receiving substrates do not comprise a growth media or hydrogel. In some embodiments, the receiving layers can comprise additional cells, for example, cells which make up the extracellular matrix. In some embodiments, a receiving substrate layer can comprise at least one, or any combination of cells selected from, but not limited to, epithelial and endothelial cells, stromal cells, fibroblast cells, adult and embryonic mesenchyme cells in the hydrogel of the receiving substrate.

Without being bound by theory, the invasion of cells in vivo occurs in the presence of other cells, which are located in specific locations in three-dimensional extracellular matrix (ECM) around the migrating cells. Presence of other cells influences migration through cell-cell contact or through factors secreted by other cells and distributed in 3D environment via diffusion.

Accordingly, in some embodiments a three-dimensional in vitro invasion assay as disclosed herein can be adapted to position two or more cell types (e.g. cancer and stroma cells) in defined positions inside physiologically-relevant 3D ECM matrix. In some embodiments, to mimic invasion of stroma, the seed-substrate layer comprising a population of cancer cells can be overlaid with receiving layers where the hydrogels comprise a population of fibroblast cells. The ability of cancer cells to invade from the seed-substrate which is layered with a receiving substrate comprising stroma cells might differ from their invasive potential where the seed-substrate which is layered with a receiving substrate where stroma cells are absent. In some embodiments, where a three-dimensional in vitro invasion assay comprises a plurality of receiving-substrates, it is envisioned that any combination of alternating layers of receiving substrates can be required. For example, a three-dimensional in vitro invasion assay can comprise at least one seed-substrate layer which is layered with a plurality of receiving layers.

In some embodiments, the receiving substrates can comprise endothelial cells, endothelial progenitor cells (EPCs) (Asahara et al, Science 275, 964-967 (1997)). In another example, endothelial progenitor cells (EPCs) can be cocultured with endothelial cells. EPCs in vivo are known to interact with vascular endothelium and undergo transendothelial migration as a first step of homing to ischemic or injured tissue. Conversely, EPC-secreted factors enhance vascularization and promote migratory response in pre-existing endothelial cells (Urbich, et al., Circ. Res. 95:343-353 (2004)). EPCs can be seeded in the hydrogels of receiving substrates and then stacked with layers that contain endothelial cell and/or seed-substrate layers to investigate long-term responses in endothelial-EPC co-culture. Cross-migration of these cells in response to angiogenic factors and oxygen concentration gradient can be investigated. In some embodiments, the receiving substrates can angiogenic factors, chemoattractants, cytokines and the like. The specific order of receiving substrates with additional cells and receiving substrates with no cells (e.g., “blank” receiving substrates) can be specifically selected to study direction of such migration and factors affecting migration of the cells. The microenvironment formed by endothelial cells can be can be examined which is also critical for proliferation and differentiation of multiple cell types. For example, a receiving substrate which comprises endothelial cells is useful to study tissue ingrowth (e.g., metastases, tumor vascularization).

In some embodiments, a substrate, e.g., a seed-substrate or receiving-substrate of a three-dimensional in vitro invasive assay as disclosed herein can be paper, e.g., filter paper, paper, polymeric, plastic, nitrocellulose, cellulose acetate, cloth or porous polymer film, or a mesh, or mesh-like substrate. One of ordinary skill in the art can select an appropriate substrate for the desired cancer cell being tested. In particular, a substrate for the seed-substrate can be different type of substrate as a substrate used as the receiving substrate. In some embodiments, a substrate may have a range of pore sizes, for example, filter paper and paper. In such embodiments, a substrate is selected where the range of pore sizes has a maximum pore size to allow a fully grown cancer cell (e.g., fully developed cancer cell) to migrate. In other embodiments, a substrate is selected where the pore size can be tightly controlled, for example, the substrate is a ceramic filter substrate, or a carbon nanotube substrate with a predefined size. In some embodiments, a substrate has an average pore size of 10-30 μm, for example, about 5 μm, or about 10 μm or about 15 μm, or about 20 μm, or about 25 μm, or about 30 μm, or larger than 30 μm. In some embodiments, a substrate used in the three-dimensional in vitro invasion as disclosed herein is selected to have a pore size to prevent accidental or incidental migration of a non-metastatic cell.

### Method of Performing the Three-Dimensional Invasive Assay

As disclosed herein, the three-dimensional invasive assay as disclosed herein can be used in a method for measuring the invasive potential of at least one population of cancer cells. In some embodiments, the invasive potential of a plurality of different populations of cancer cells can be assayed at one time, allowing for a high-capacity and high-throughput invasive assay. In such embodiments, a biological sample comprising a population of cells is placed at a location of a hydrogel of a seed substrate. As disclosed herein, the methods for performing a high-capacity invasive assay as disclosed herein can be automated, e.g., for example, using a system as disclosed herein. In some embodiments, the system is a robotics system.

Advantages of a three-dimensional in vitro invasive assay as disclosed herein, and the methods of the invention is that they can be performed rapidly, with high-capacity and on a cost effective basis without having to wait for a metastasis to occur in the subject. The assay and methods as disclosed herein is efficient, in that an investigator can readily compare a variety of different populations of cancer cells from one or more tumor sites in a subject, and with cancer cells with known metastatic potential and invasive capacity. In embodiments three-dimensional in vitro invasive assay as disclosed herein can be conducted economically (e.g., using multi-patterned seed substrates that require minimal use of the agent to be tested and inexpensive cell culture methods and materials). Such methods using a three-dimensional in vitro invasive assay as disclosed can be readily adapted to high throughput methods, e.g. using robotic or other automated procedures and computerized systems as disclosed herein. Furthermore, by selecting a variety of reference cancer cell populations (e.g. having a defined degree of metastatic potential or invasive capacity or a preference for a specific site of metastasis), the method can be used to identify the degree of metastatic potential and invasive capacity of a large number of cancer cell populations at one time. In some embodiments, the three-dimensional in vitro invasive assay as disclosed herein can also be used to identify a variety of anti-metastatic agents, which affect different stages of metastasis, and/or which are directed against metastases to particular tissues.

In some embodiments the seed-substrate can be immediately layered and contacted with at least one receiving substrate to form a three-dimensional cellular invasive assay and incubated for a pre-determined time as required for the migration of cells in the invasive assay. In some embodiments, the cancer cells are cultured on the seed-substrate for a pre-determined time prior to being contacted with at least one receiving layer to form the three-dimensional cellular invasive assay. In such embodiments, the cells can be cultured on the seed-substrate for a pre-defined period of time, for example, for at least about 1 hour, or at least about 2 hours, or at least about 6 hours, or at least about 12 hours, or at least about 24 hours, or at least about 48 hours, or at least about 3 days, or at least about 5 days, or at least about 7 days, or at least about 14 days, or any time between 1-48 hours, or between about 3-14 days. In some embodiments, a seed-substrate is cultured for about 24 hours, or about 48 hours or about 72 hours prior to be being combined with at least one receiving substrate to form a three-dimensional cellular invasive assay.

As disclosed above, a seed-substrate (comprising cancer cells) is layered on at least one, or a plurality of upper or lower receiving substrates to form a three-dimensional invasive cellular assay, and is incubated for a pre-determined period of time. In some embodiments, the three-dimensional invasive cellular assay is incubated in an appropriate culture media. In some embodiments, the three-dimensional assay is cultured in the absence of a culture media. In some embodiments, the three-dimensional assay as disclosed herein can be cultured in vivo, where the three-dimensional substrate is implanted into an animal model, or tissue explant. In some embodiments, the pre-determined incubation time is determined by one of ordinary skill in the art to allow the cancer cells in the seed-substrate to migrate to one or more receiving layers. In some embodiments, the pre-determined period of time is between 2-6 hours, or between 6-12 hours, or between 12-24 hours, or between 24-48 hours, or between 48-36 hours or more than 36 hours. In some embodiments, the pre-determined period incubation time is at least about 2 hours, or at least about 4 hours, or at least about 6 hours, or at least about 12 hours, or at least about 24 hours, or at least about 48 hours, or at least about 3 days, or at least about 5 days, or at least about 7 days, or at least about 14 days, or any time between about 2-48 hours, or between 3-5 days, or between about 3-14 days. In some embodiments, a three-dimensional in vitro invasive assay can be incubated for about 24 hours, or about 2 days, or about 3 days or about 4 days or about 5 days.

After a pre-determined period of time after seeding and incubation of the three-dimensional invasive assay, the seed-substrate layer is separated from one or more of the receiving substrate layers and the quantity of the cells on one or more receiving layers is measured. The presence or detection of cancer cells that have entered one or more receiving substrate layers of the three-dimensional assay after the pre-defined incubation period is indicative of the invasion of the cancer cell and the metastatic potential of the cancer. The presence of cancer cells in one or more receiving substrate layers indicates that the cancer cells from the seed-substrate layer are capable of invasion and indicates the cancer cell has a metastatic potential and invasive capacity.

The seed-substrates and at least one receiving substrate are stacked, placed in cell culture medium for a pre-determined incubation time, and then destacked. In particular embodiments, upon destacking or separating the seed-substrate from each of the receiving substrates, the cells in the separated substrates remain viable and can be cultured separately or characterized using any assays described herein to compare the number of cells in each receiving substrate, and optionally, in comparison with the number of cells from the seed-substrate layer.

The number of cells in each layer prior and post-migration can be quantified as described above. The quantity of the cancer cells in the receiving layer can be measured by any method commonly known by one of skill in the art, e.g., by imaging or other quantitative methods, e.g., PCR amplification etc.

In some embodiments, the amount of cells on the outer most receiving substrate layer (e.g., the receiving layer furthest from the seed-substrate) is measured. In some embodiments, the amount cells on all the receiving layers are measured. In some embodiments, cells on alternating receiving substrate layers are measured. In some embodiments, the amount of cells is measured on at least 1, or at least 2, or at least 3, or at least 4, or at least 5, or at least about 6, or at least about 7, or at least about 8, or at least about 9, or at least about 10, or at least about 12, or at least about 15, or more than 15 receiving layers. In some embodiments, cells are measured on the receiving substrate which contacts the seed-substrate layer and also on the outer most receiving substrate layer (e.g., the receiving layer furthest from the seed-substrate). The cells can be measured on any combination of upper and/or lower receiving layers.

Additionally, in some embodiments, the cells can be measured on one or more seed-substrate layers. In some embodiments, where it is desirable to compare the amount of cells before and post-migration, the amount of cells can be measured on the seed-substrate prior stacking the seed-substrate on a receiving substrate and prior to the incubation period (e.g., before the assay has begun), and then measuring the amount of cells on the same seed-substrate after it is separated from the receiving substrate after completion of the incubation period.

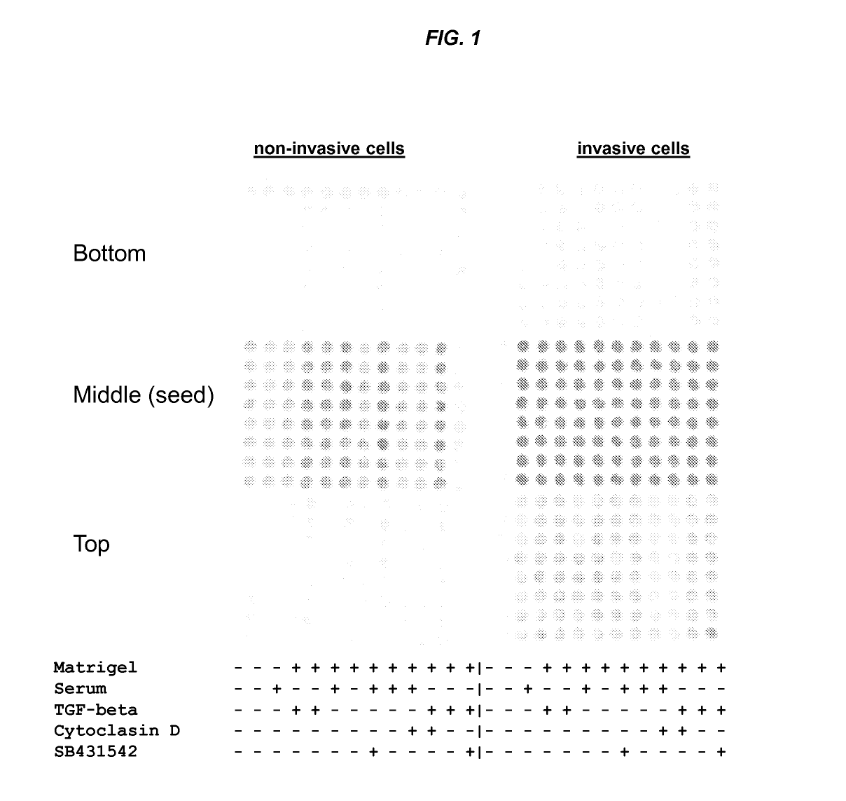

In some embodiments, viable cells in each substrate can be quantified, using cell proliferation reagents (e.g., Alamar Blue) or fluorimetric assays (e.g., calcein stain, FIG. 1). Alternatively, in some embodiments, cells in each substrate layer can be fixed and quantified with fluorescent labeling agents (e.g. SYTOX to label DNA, Phalloidine to label F-actin). For example, to qualitative and quantitative measure or detect the presence of cells in each separated substrate, one can use any method commonly known in the art such as image analysis of the substrate, for example, such as using any conventional method (fluorescent microscope, fluorescent or luminescence scanner, phosphorimager, gel imager, colorimetric imaging etc).

In alternative embodiments, cells in each substrate layer can be lysed, and this lysate can be used to measure the level of expression of genes of interest (e.g. a house keeping gene such as actin, or VEGF and IGFPB3). In some embodiments, one can also measure the cellular concentration of specific proteins (e.g. hypoxia induced factors (HIF) 1 and 2 with commercial ELISA kits). The cells can be isolated from the substrates using any known method, e.g., enzymatically. One exemplary enzyme is reactive to cellulose substrates, such as cellulase from Trichoderma reesei.

The level of gene expression of cells on each substrate layer can be analyzed, for example, after lysis and removal from the substrate and the lysates can be used in standard assays known in the art, e.g., Northern analysis, ribonuclease protection assays, or reverse transcription-polymerase chain reaction (RT-PCR) (see, e.g., Sambrook et al., Molecular Cloning: A Laboratory Manual (3rd ed. 2001)). In some embodiments, gene expression can be measured by methods selected from the group comprising; reverse-transcription polymerase chain reaction (RT-PCR) or by quantitative RT-PCR (QRT-PCR) reaction.

Changes in gene expression can be assayed using known genome-wide analysis techniques. For example, an Affymetrix GeneChip® can be used to perform transcriptome analysis; Illumina Deep Sequencing can be used to perform the analysis of coding and regulatory RNAs (e.g. micro RNA or miRNA) Gene expression and regulatory RNA profiles can be compared to known profiles for cells grown ex vivo and in vivo. In some embodiments, expression profiling and flow cytometry analysis of the migrated cells on the receiving substrates can be used to characterize the migrated cells differentiation state.

The cellular material obtained from the cells cultured in three- dimensional cellular arrays can also be used to analyze protein levels, by methods such as by Western analysis or immunoassays. Proteins, carbohydrates and metabolites from the cellular material can be analyzed by known global profiling methods such as those based on mass spectrometry (e.g. shotgun proteomics, metabolomics) or NMR spectroscopy. In some embodiments, protein expression can be measured by a method selected from the group consisting of; immunoblot analysis, immunohistochemical analysis; ELISA, isoform-specific chemical or enzymatic cleavage, protein array or mass spectrometry. In some embodiments, protein expression can be measured by contacting the biological sample with at least one protein binding agent selected from the group consisting of; antibodies; recombinant antibodies, chimeric antibodies, tribodies, midibodies, protein-binding agents, small molecule, recombinant protein, peptides, aptamers, avimers and derivatives or fragments thereof.

In some embodiments, cells can be enzymatically removed from each substrate layer and characterized using standard cytometry assays, e.g., by flow-cytometry analysis, heamocytmeter, automated cell counter etc. Such flow-cytometry methods are useful where it is desirable to assess the number of live, apoptotic, and necrotic cells and to perform cell cycle analysis of cell population in each substrate layer. In some embodiments, fluorescent reporters or cell tracer dyes can be used to label the cells in each substrate layer and/or to trace the origin of each cell in each layer. For example, in some embodiments, cancer cells in a biological sample can be labeled with a fluorescent marker, e.g., a fluorescent labeled antibody to a specific cell-surface marker to characterize and trace the migration of specific cell types into the receiving substrate layers.

In some embodiments, a comparative transcriptome profiling of the cancer cells isolated from different layer sheet can provide novel insight on transformations that occur in the interior of solid tumors and other non-vascularized three-dimensional structures composed of multiple layers of cells.

Certain embodiments will employ conventional techniques of molecular biology (including recombinant techniques), microbiology, cell biology, biochemistry, and immunology, which are known to those of ordinary skill of the art. Such techniques are described in, e.g., “Molecular Cloning: A Laboratory Manual”, third edition (Sambrook et al, 2001); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

In some embodiments, cells in each substrate layer (e.g., seed-substrate and/or receiving substrate) can be automatically determined using automated analysis software, which can optionally include data analysis of the number of cells on each substrate layer.

### Determining Metastatic Potential of Cancer Cells.

As disclosed herein, the present invention provides methods to assess the metastatic potential of a population of cancer cells using a three dimensional in vitro invasive assay as disclosed herein. After incubation of the three dimensional in vitro invasive assay for a predetermined period of time, the seed-substrate layer is separated from each of the receiving substrate layers and the amount of cells on one or more of the receiving substrate layers (and optionally the seed-substrate layer) are quantified by measuring the amount of cells by any of the methods described above to determine the migration of the cells from the seed-substrate layer.

The amount of migration of the cancer cells from the seed-substrate onto one or more receiving substrates is a function of (e.g., proportional to) the metastatic potential (ability, activity, capacity) of the cell; and wherein a significant amount of cancer cells detected on one or more receiving substrate indicates that the cancer cell population comprises cancer metastatic cells and the cancer cell population has invasive capacity. The amount of cancer cell migration into one or more receiving substrate layers can be determined by measuring the amount of cells present, e.g., using imaging (e.g., detection of a fluorescent marker or luciferase) or other quantitative methods such as gene expression as disclosed herein.

The invasive potential of a cancer cell population can be determined a variety of different ways using a variety of different parameters. Exemplary methods to determine the invasive potential of a cancer cell population are disclosed herein, however, other calculations are encompassed in the present invention.

In some embodiments, the invasive potential is determined by measuring the amount of cells on a predetermined receiving substrate. In such embodiment, the presence of cells on a receiving substrate which is above a predetermined level of accidental migration indicates the cancer cell population comprises a population of metastatic cancer cells and cancer cells with invasive population.

In another embodiment, a representative calculation to determine the metastatic potential of a population of cancer cells can be based on % of migration of cancer cells from the seed-substrate layer. In such an embodiment, a seed-substrate can be seeded with a predefined number of cancer cells (e.g., total starting population). After a predefined time of incubation, the amount of cells on one or more receiving substrates (e.g., the migrated population) can be calculated as a % of the total starting population of cancer cells on the seed-substrate. In some embodiments, the % of cells which have migrated (% migrated cells) is a measure of the invasive capacity of the cancer cell population and is predictive of the metastatic potential of the cancer cell population. In some embodiments, where at least about 5%, or at least about 10% or more than 10% of the total cell population is present on one or more receiving substrates, it indicates the population of cells has cancer cells with invasive capacity and is predictive of the metastatic potential of the cancer cell population. In some embodiments, if the % migrated cells ranges between 0-4% migrated cells, it indicates a negligible invasive capacity and very low risk of the cell population comprising metastatic cancer cells, whereas a 5-10% range of migrated cells indicates a low level invasive capacity and low risk of the cell population comprising metastatic cancer cells, whereas a 11-20% range of migrated cells indicates a medium level invasive capacity and medium risk of the cell population comprising metastatic cancer cells, and where a % migrated cells above 20% indicates a high level invasive capacity and high risk of the cell population comprising metastatic cancer cells and predicts the cancer is a metastatic cancer. The number of migrated cells depends on the number of days over which migration occurs. For example, in some embodiments, a migration of 11-20% of cells over a 3-5 day migration period indicates a medium level invasive capacity and medium risk of the cell population comprising metastatic cancer cells, and whereas a migration of above 20% of cells over a 3-5 day period indicates a high level invasive capacity and high risk of the cell population comprising metastatic cancer cells and predicts the cancer is a metastatic cancer. In such embodiments, a less than 11% migration of cells over a 3-5 day period indicates that the cells are non-invasive cells, and have a low to negligent risk of the cells comprising metastatic cancer cells.

In some embodiments, a migration of <5-10% of cells, e.g., between about 5-10%, or about 5%, or about 6%, or about 7%, or about 8% or about 9%, or about 10%, over a time period of 1-2.9 days migration period indicates a medium level invasive capacity and medium risk of the cell population comprising metastatic cancer cells, and whereas a migration of above 10% of cells over a time period of 1-2.9 day period indicates a high level invasive capacity and high risk of the cell population comprising metastatic cancer cells and predicts the cancer is a metastatic cancer. In such embodiments, a less than 10% migration of cells over a 1-2.9 day period indicates that the cells are non-invasive cells, and have a low to negligent risk of the cells comprising metastatic cancer cells.

In some embodiments, after separation of the seed-substrate from one or more receiving substrates, the measured quantity of cells on the receiving substrate (e.g., the migrated cells) can be compared to the measured quantity of cells on the seed substrate after a pre-determined incubation period, where the quantity of migrated cells on a receiving substrate can be calculated as a % of the quantity of cells measured on the seed-substrate.

In further embodiments, the amount of cells measured on the seed-substrate after a predetermined incubation period (e.g., the non-migrating population) can also be compared to the amount of cells present on the seed-substrate prior to stacking with a receiving substrate (e.g., a comparison of the total starting population of cells and/or a predetermined number of cancer cells with the number of cells on the seed-substrate layer post-migration). In some embodiments, a decrease of about 5%, or about 10%, or about 20% of cells, or a decrease of more than 20%, of cells on the seed-substrate layer post-migration as compared to the total starting population of cells (e.g., a the predetermined number of cells or the measured cells on the seed-substrate prior to the incubation period) is indicative that the population of cancer cells comprises cancer cells with invasive capacity and is predictive of the metastatic potential of the cancer cell population. In some embodiments, where a decrease of about 0-4% of cells on the seed-substrate post-incubation (as compared to the total starting population on the seed-substrate pre-incubation) indicates a negligible invasive capacity and very low risk of the cell population comprising metastatic cancer cells, whereas a 5-10% decrease of cells on the seed-substrate post-incubation (as compared to the total starting population on the seed-substrate pre-incubation) indicates a low level invasive capacity and low risk of the cell population comprising metastatic cancer cells, whereas a 11-20% decrease of cells on the seed-substrate post-incubation (as compared to the total starting population on the seed-substrate pre-incubation) indicates a medium level invasive capacity and medium risk of the cell population comprising metastatic cancer cells, and where a decrease greater than 20% of cells on the seed-substrate post-incubation (as compared to the total starting population on the seed-substrate pre-incubation) indicates a high level invasive capacity and high risk of the cell population comprising metastatic cancer cells and predicts the cancer is a metastatic cancer.

It is envisioned that in some embodiments, where the amount of cells measured on the seed-substrate prior to incubation (e.g., total starting population) is compared to the amount of cells on another substrate after incubation, e.g., the amount of cells measured on the seed-substrate after incubation (e.g., the non-migrating population), or the amount of cells measured on a receiving substrate (e.g., the migrated population), that cell proliferation of the cells on the seed-substrate that occurs during the incubation period should be taken into account and factored into the calculation, and/or adequate controls established to measure such proliferation.

In another embodiment, a representative calculation to determine the metastatic potential of a population of cancer cells can be based on % of migration as compared to a reference cancer cell line. In some embodiments, a reference cancer cell line is a cancer cell line of known metastatic potential and invasive capacity. In some embodiments, a positive reference cancer cell line is a high-metastatic cancer cell line, for example but not limited to, a breast cancer cell line such as MDA-MB-231, or a prostate cancer cell line such as highly invasive PC-3 and DU-154 metastatic cancer cell lines isolated from bone and brain metastases. Other highly metastatic reference cell lines can also be used, for example, LM2, HeLa cells, known carcinoma and sarcoma cell lines, lung adrenocarcinoma line Anip 973, breast cancer cell lines MDA-MB-468 and MDA-MB-435, human glioblastoma line 324, mouse melanoma B16 among others. In some embodiments, a positive reference cancer cell line is a low-metastatic cancer cell line, for example but not limited to, the weakly invasive cancer cell lines LNCaP and CWR-22 lines. In some embodiments, a reference cell line is a negative reference cancer cell line, such as a normal cell line, for example but not limited to, normal prostate epithelial cells (RWPE-1) and fibroblasts (H₁N 3T3).

In another embodiment, a representative calculation to determine the metastatic potential of a test population of cancer cells can be based on measuring the quantity of cells on at least one receiving substrate and on comparing the amount of migrated cells from the biological sample comprising the test cancer cell population with the amount of cells migrated from the reference cell population, where the amount of reference cells on the receiving substrate sets a baseline for the level of metastatic potential and invasive capacity. Accordingly, in such embodiments both positive and/or negative reference cell populations can serve as internal controls on the receiving substrate as a measure of a cancer cells invasive capacity.

As an exemplary example, assume that that the amount of reference cells of a positive highly-metastatic reference cancer cell population measured on a receiving substrate is set an arbitrary value of 10, and the amount of reference cells of a negative reference cell population measured on a receiving substrate is set an arbitrary value of 0, which establishes a scale, or an “invasive scale” for an invasive potential of a cancer cell line. Accordingly, the amount of cells on the receiving substrate can be directly compared to the amount of cells from a positive reference cell line and/or optionally from a negative reference cell line. In some embodiments, a positive reference cell population of a low-metastatic cell line can be used to establish a scale of invasive potential of a cancer cell population. A comparison of the amount of cancer cells of the test cancer cell population on the receiving substrate (e.g., migrating population) with the amount of cells of positive reference cell line (e.g., a highly-invasive population or a low-invasive population) and/or a negative cell line (e.g., a non-migrating population) can be used to determine the level of invasive potential of a test cancer cell population.

For example, where the amount of cells of the test cancer cell population on the receiving substrate is greater than the amount of cells from the positive reference cell population, the test cell population will have an arbitrary value of greater than 10, and will be indicative of a highly metastatic cancer cell population. Similarly, where the amount of cells of the test cancer cell population on the receiving substrate is less than the amount of cells from the (highly-metastatic) positive reference cell population, but more than the amount of cells from the negative reference cell population, the test cancer cell population will have an arbitrary value of greater than 0 but less than 10, and will be indicative of a metastatic cancer cell population. In some embodiments, the amount of cells of the test cancer cell population on the receiving substrate can be compared to the amount of cells from a low-metastatic positive reference cell population and scaled accordingly.

In some embodiments, the amount of cells of the test cancer cell population on the receiving substrate can be compared as a % (or fold increase or decrease) of the amount of cells of a positive (both highly-metastatic and/or low-metastatic) reference cell population and/or a % (or fold-increase) of the amount of cells of a negative reference cell population on the receiving substrate. For example, where there are at least about 20%, or at least about 30%, or at least about 40% of amount of cells of the test cancer cell population on the receiving substrate as compared to the amount of cells of highly- invasive positive reference cell line, it is indicative of a low metastatic potential and invasive capacity of the test cancer cell population, whereas where there are at least about 50%, or at least about 60%, or at least about 70%, or at least about 80%, or at least about 90%, or at least about 95%, or at least about 100%, or more than 100%, e.g., at least about 1.2-fold, or about 1.5-fold or 2-fold, or 3-fold or more than 3-fold of amount of cells of the test cancer cell population on the receiving substrate as compared to the amount of cells of highly-invasive positive reference cell line, it is indicative of a high metastatic potential and high invasive capacity of the test cancer cell population.

In another embodiment, a representative calculation to determine the metastatic potential of a test population of cancer cells is an “invasive index” and is expressed as the ratio of the % migration of the test population of cancer cells over the % migration of a positive reference cancer cell line.

To determine the % migration, a representative calculation is as follows:

$$\% migration = \frac{ \begin{matrix}Mean number of cells on the \\ receiving substrate ( migrating cells )\end{matrix}}{\begin{matrix}mean number of cells on the seed \text{-} \\ substrate post incubation ( non \text{-} migrating cells )\end{matrix}}$$

To determine the Invasive Index, a representative calculation is as follows:

$$Invasion index = \frac{\% migration of test cancer c ell population}{\% migration of positive reference cancer cell line} .$$

Additionally in some embodiments, a negative reference cell population (e.g., a negative control) can be a positive reference cell line (e.g., a highly-metastatic and/or low-metastatic reference cell population) which has been combined (e.g., in the hydrogel of the seed substrate and/or receiving substrate) with an inhibitor of actin-based motility. Such inhibitors of actin-based mobility are well known in the art, and include for example, cytoclasin D and the like. Other negative controls also include the test population of cancer cells combined with an inhibitor of actin-based motility.

In some embodiments, one can use other calculations, such as, but not limited to, % of cancer cells with specific marker that migrated from layer to layer; % of cells that migrate in a 3D construct (tumor) of specific geometry; and % of cells that migrate inside specific location of the 3D construct.

### Computer Systems

One aspect of the present invention relates to a computerized system for processing the high-capacity three-dimensional in vitro invasion assay and generating a measure of the metastatic potential and invasive capacity of a cancer cell population. The computer system can include: (a) at least one memory containing at least one computer program adapted to control the operation of the computer system to implement a method that includes: (i) receiving data of the amount of cells on receiving substrate e.g., the amount of cells (e.g., test cancer cells and/or reference cancer cells) on one or more receiving substrates; (ii) receiving data of the measured amount of cells on a seed substrate e.g., the amount of cells (e.g., test cancer cells and/or reference cancer cells) on one or more seed substrates; (iii) generating a report of the invasive potential of the test cancer cell population based on the comparison of the data of the test cancer cell line and a reference cancer cell line; and (b) at least one processor for executing the computer program.

In some embodiments, a computer program is adapted to control the operation of the computer system to implement a method that further includes at least one of: (i) receiving data from the receiving substrate, (ii) receiving data from a seed substrate prior to incubation, and (iii) receiving data from a seed-substrate after incubation; (iii) generating a report of the invasive potential of the test cancer cell population based on the comparison of the data of the test cancer cell line and a reference cancer cell line.

Another aspect of the present invention relates to a computer readable medium comprising instructions, such as computer programs and software, for controlling a computer system to process the data from the measured receiving layers and/or measured seed layers and generate a report of the invasive capacity and metastatic potential of a test cancer cell population, comprising any one, or a combination of calculations selected from: (i) % of cancer cells with specific marker that migrated from layer to layer; (ii) % of cells that migrate in a 3D construct of specific geometry; (iii) % of cells that migrate inside specific location of the 3D construct. In some embodiments, the three-dimensional assay can be performed in the presence or absence of chemotherapeutic agents, and can report (i) % of cancer cells with specific marker that migrated from layer to layer in the presence or absence of chemotherapeutic agent (ii) % of cells that migrate in a 3D construct of specific geometry in the presence or absence of chemotherapeutic agent, and (iii) % of cells that migrate inside specific location of the 3D construct in the presence or absence of chemotherapeutic agent.

The computer system can include one or more general or special purpose processors and associated memory, including volatile and non-volatile memory devices. The computer system memory can store software or computer programs for controlling the operation of the computer system to make a special purpose computer system according to the invention or to implement a system to perform the methods and analysis according to the invention.

In some embodiments, a computer system can include, for example, an Intel or AMD x86 based single or multi-core central processing unit (CPU), an ARM processor or similar computer processor for processing the data. The CPU or microprocessor can be any conventional general purpose single- or multi-chip microprocessor such as an Intel and AMD processor, a SPARC processor, or an ARM processor. In addition, the microprocessor may be any conventional or special purpose microprocessor such as a digital signal processor or a graphics processor. The microprocessor typically has conventional address lines, conventional data lines, and one or more conventional control lines. As described below, the software according to the invention can be executed on dedicated system or on a general purpose computer having a DOS, CPM, Windows, Unix, Linix or other operating system. The system can include non-volatile memory, such as disk memory and solid state memory for storing computer programs, software and data and volatile memory, such as high speed ram for executing programs and software.

Computer-readable physical storage media useful in various embodiments of the invention can include any physical computer-readable storage medium, e.g., solid state memory (such as flash memory), magnetic and optical computer-readable storage media and devices, and memory that uses other persistent storage technologies. In some embodiments, a computer readable media can be any tangible media that allows computer programs and data to be accessed by a computer. Computer readable media can include volatile and nonvolatile, removable and non-removable tangible media implemented in any method or technology capable of storing information such as computer readable instructions, program modules, programs, data, data structures, and database information. In some embodiments of the invention, computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks), Blue-ray, USB drives, micro-SD drives, or other optical storage media, magnetic cassettes, magnetic tape, magnetic disk storage or other magnetic storage media, other types of volatile and non-volatile memory, and any other tangible medium which can be used to store information and which can read by a computer including and any suitable combination of the foregoing.

The present invention can be implemented on a stand-alone computer or as part of a networked computer system. In a stand-alone computer, all the software and data can reside on local memory devices, for example an optical disk or flash memory device can be used to store the computer software for implementing the invention as well as the data. In alternative embodiments, the software or the data or both can be accessed through a network connection to remote devices. In one embodiment, the invention can use a client-server environment over a network, e.g., a public network such as the internes or a private network to connect to data and resources stored in remote and/or centrally located locations. In this embodiment, a server such as a web server can provide access, either open access, pay as you go or subscription based access to the information provided according to the invention. In a client server environment, a client computer executing a client software or program, such as a web browser, connects to the server over the network. The client software provides a user interface for a user of the invention to input data and information and receive access to data and information. The client software can be viewed on a local computer display or other output device and can allow the user to input information, such as by using a computer keyboard, mouse or other input device. The server executes one or more computer programs that receives data input through the client software, processes data according to the invention and outputs data to the user, as well as provide access to local and remote computer resources. For example, the user interface can include a graphical user interface comprising an access element, such as a text box, that permits entry of data from the assay, e.g., the data from a positive reference cancer cell, as well as a display element that can provide a graphical read out of the results of a comparison with a cancer cell with a known metastatic potential or invasive capacity, or data sets transmitted to or made available by a processor following execution of the instructions encoded on a computer-readable medium.

Embodiments of the invention also provide for systems (and computer readable medium providing instructions for causing computer systems) to perform a method for determining quality assurance of a pluripotent stem cell population according to the methods as disclosed herein.

In some embodiments of the invention, the computer system software can include one or more functional modules, which can be defined by computer executable instructions recorded on computer readable media and which cause a computer to perforin, when executed, a method according to one or more embodiments of the invention. The modules can be segregated by function for the sake of clarity, however, it should be understood that the modules need not correspond to discreet blocks of code and the described functions can be carried out by the execution of various software code portions stored on various media and executed at various times. Furthermore, it should be appreciated that the modules can perform other functions, thus the modules are not limited to having any particular function or set of functions. In some embodiments, functional modules are, for example, but are not limited to, an array module, a determination module, a storage module, a reference comparison module, a normalization module, and a display module to display the results (e.g., the invasive potential of the test cancer cell population). The functional modules can be executed using one or multiple computers, and by using one or multiple computer networks.

The information embodied on one or more computer-readable media can include data, computer software or programs, and program instructions that as a result of being executed by a computer, transform the computer to a special purpose machine and can cause the computer to perform one or more of the functions described herein. Such instructions can be originally written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied can reside on one or more of the components of a computer system or a network of computer systems according to the invention.

In some embodiments, a computer-readable media can be transportable such that the instructions stored thereon can be loaded onto any computer resource to implement the aspects of the present invention discussed herein. In addition, it should be appreciated that the instructions stored on computer readable media are not limited to instructions embodied as part of an application program running on a host computer. Rather, the instructions may be embodied as any type of computer code (e.g., object code, software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

In some embodiments, a system as disclosed herein, can receive data of cells on a receiving substrate or cells on a seed-substrate from any method of determining the amount or cells. Where the amount of cells is measured by protein expression, the system as disclosed herein can be configured to receive data from an automated protein analysis systems, for example, using immunoassay, for example western blot analysis or ELISA, or a high through-put protein detection method, for example but are not limited to automated immunohistochemistry apparatus, for example, robotically automated immunodetection apparatus which in an automated system to separate the receiving substrate and the seed-substrate layers, perform immunohistochemistry procedure and detect intensity of immunostaining, such as intensity of an antibody staining of the substrates and produce output data. Examples of such automated immunohistochemistry apparatus are commercially available, and can be readily adapted to automatically detect the amount of cells on the separated substrates of the in vitro invasion assay as disclosed herein, and include, for example but not limited to such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx 6000™, for Drug Discovery labs. Both systems BioGenex systems perform “All-in-One, All-at-Once” functions for cell and tissue testing, such as Immunohistochemistry (IHC) and In Situ Hybridization (ISH).

In some embodiments, a system as disclosed herein, can receive data of cells on a receiving substrate or cells on a seed-substrate from an automated ELISA system (e.g. DSX® or DK® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters), or adapted systems thereof for detecting cells on the separated substrates as disclosed herein.

In some embodiments, a system as disclosed herein, can receive data of cells on a receiving substrate or cells on a seed-substrate from any method of determining the amount or cells. Where the amount of cells is measured by gene expression, the system as disclosed herein can be configured to receive data from an automated gene expression analysis system, e.g., an automated protein expression analysis including but not limited Mass Spectrometry systems including MALDI-TOF, or Matrix Assisted Laser Desorption Ionization—Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array.

In some embodiments of the present invention, an automated gene expression analysis system can record the data electronically or digitally, annotated and retrieved from databases including, but not limited to GenBank (NCBI) protein and DNA databases such as genome, ESTs, SNPS, Traces, Celara, Ventor Reads, Watson reads, HGTS, etc.; Swiss Institute of Bioinformatics databases, such as ENZYME, PROSITE, SWISS-2DPAGE, Swiss-Prot and TrEMBL databases; the Melanie software package or the ExPASy WWW server, etc., the SWISS-MODEL, Swiss-Shop and other network-based computational tools; the Comprehensive Microbial Resource database (The institute of Genomic Research). The resulting information can be stored in a relational data base that may be employed to determine homologies between the reference data or genes or proteins within and among genomes.

In some embodiments, the data of amount cells on a receiving substrate and/or amount of cells on a seed-substrate can be received from a memory, a storage device, or a database. The memory, storage device or database can be directly connected to the computer system retrieving the data, or connected to the computer through a wired or wireless connection technology and retrieved from a remote device or system over the wired or wireless connection. Further, the memory, storage device or database, can be located remotely from the computer system from which it is retrieved.

Examples of suitable connection technologies for use with the present invention include, for example parallel interfaces (e.g., PATA), serial interfaces (e.g., SATA, USB, Firewire,), local area networks (LAN), wide area networks (WAN), Internet, Intranet, and Extranet, and wireless (e.g., Blue Tooth, Zigbee, WiFi, WiMAX, 3G, 4G) communication technologies

Storage devices are also commonly referred to in the art as “computer-readable physical storage media” which is useful in various embodiments, and can include any physical computer-readable storage medium, e.g., magnetic and optical computer-readable storage media, among others. Carrier waves and other signal-based storage or transmission media are not included within the scope of storage devices or physical computer-readable storage media encompassed by the term and useful according to the invention. The storage device is adapted or configured for having recorded thereon cytokine level information. Such information can be provided in digital form that can be transmitted and read electronically, e.g., via the Internet, on diskette, via USB (universal serial bus) or via any other suitable mode of communication.

As used herein, “stored” refers to a process for recording information, e.g., data, programs and instructions, on the storage device that can be read back at a later time. Those skilled in the art can readily adopt any of the presently known methods for recording information on known media to contribute to the data of the amount of cells on a receiving substrate and/or amount of cells on a seed-substrate a reference scorecard data, e.g., data of an amount of cells on a receiving substrate from a biological sample obtained from the same subject (e.g., obtained from the subject at an earlier timepoint) and/or amount of cells from one or more reference cancer cell lines as disclosed in the methods herein.

A variety of software programs and formats can be used to store the scorecard data and information on the storage device. Any number of data processor structuring formats (e.g., text file or database) can be employed to obtain or create a medium having recorded scorecard thereon.

In some embodiment, the system has a processor for running one or more programs, e.g., where the programs can include an operating system (e.g., UNIX, Windows), a relational database management system, an application program, and a World Wide Web server program. The application program can be a World Wide Web application that includes the executable code necessary for generation of database language statements (e.g., Structured Query Language (SQL) statements). The executables can include embedded SQL statements. In addition, the World Wide Web application can include a configuration file which contains pointers and addresses to the various software entities that provide the World Wide Web server functions as well as the various external and internal databases which can be accessed to service user requests. The Configuration file can also direct requests for server resources to the appropriate hardware devices, as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In one embodiment, the system as disclosed herein can be used to compare the data of the amount of population of test cancer cells on a receiving substrate and/or on a seed-substrate with the data of the amount of population of reference cancer cell line (e.g. a positive highly- or low-metastatic cell line, or a negative cell line) on a receiving substrate and/or on a seed-substrate. For example, the system can receive onto its memory data of the amount of population of test cancer cells on a receiving substrate and/or on a seed-substrate and compare it with one or more stored reference cancer cell lines, or compare with one or more data of the test cancer cell population previously analyzed at an earlier timepoint.

In some embodiments of this aspect and all other aspects of the present invention, the system can compare the data in a “comparison module” which can use a variety of available software programs and formats for the comparison operative to compare sequence information determined in the determination module to reference data. In one embodiment, the comparison module is configured to use pattern recognition techniques to compare sequence information from one or more entries to one or more reference data patterns. The comparison module may be configured using existing commercially-available or freely-available software for comparing patterns, and may be optimized for particular data comparisons that are conducted. The comparison module can also provide computer readable information related to the level or amount of invasion of a cell into a receiving substrate and the like as disclosed herein, including but not limited to (i) % of cancer cells with specific marker that migrated from layer to layer; (ii) % of cells that migrate in a 3D construct of specific geometry; (iii) % of cells that migrate inside specific location of the 3D construct.

In some embodiments of the invention, the system comprises comparison software which is used to determine whether the data of the amount of population of test cancer cells on a receiving substrate and/or on a seed-substrate falls outside a normal range of a negative control reference cell line, e.g., is higher than a negative control. In one embodiment, where the data of the amount of population of test cancer cells on a receiving substrate is higher by a statically significantly amount above a negative reference cell line it indicates likelihood of invasive capacity and that the test cancer cell population comprises a metastatic cancer cell population. In such instances, the software can be configured to indicate or signal that the test cancer cell population comprises a metastatic cancer cell population will likely result in a metastatic cancer in a subject.

In some embodiments, the system comprises comparison software used to determine whether the data of the amount of population of test cancer cells on a receiving substrate and/or on a seed-substrate falls within a normal range of for positive reference cell line, for example, higher than a positive low-metastatic reference cell line or the same level or higher than a highly-metastatic reference cell line. In one embodiment, where the data of the amount of population of test cancer cells on a receiving substrate is higher by a statically significantly amount above a low-metastatic positive reference cell line it indicates low likelihood of invasive capacity and that the test cancer cell population comprises a low-metastatic cancer cell population. In some embodiments, where the data of the amount of population of test cancer cells on a receiving substrate is within a range similar, or higher by a statically significantly amount to a high-metastatic positive reference cell line it indicates high likelihood of invasive capacity and that the test cancer cell population comprises a highly-metastatic cancer cell population. In such instances, the software can be configured to indicate or signal that the test cancer cell population comprises a metastatic cancer cell population will likely result in a metastatic cancer in a subject.

By providing data of the amount of population of test cancer cells on a receiving substrate and/or on a seed-substrate in computer-readable form, one can use the data to compare with data of the amount of population of reference cancer cells on a receiving substrate and/or on a seed-substrate within the storage device. For example, search programs can be used to identify relevant reference data (i.e. data of appropriate reference cancer cell lines) that match the same type of cancer as the cancer of the test cancer cell population. The comparison made in computer-readable form provides computer readable content which can be processed by a variety of means. The content can be retrieved from the comparison module, the retrieved content.

In some embodiments, the comparison module provides computer readable comparison result that can be processed in computer readable form by predefined criteria, or criteria defined by a user, to provide a report which comprises content based in part on the comparison result that may be stored and output as requested by a user using a display module. In some embodiments, a display module enables display of a content based in part on the comparison result for the user, wherein the content is a report indicative of the results of the comparison of the test cancer cell population with a reference positive cancer cell line (e.g., highly-metastatic cancer cell line or low-metastatic cancer cell line) or a negative reference cell line.

In some embodiments, the display module enables display of a report or content based in part on the comparison result for the end user, wherein the content is a report indicative of the results of the comparison of the test cancer cell population with a reference positive cancer cell line (e.g., highly-metastatic cancer cell line or low-metastatic cancer cell line) or a negative reference cell line.

In some embodiments of this aspect and all other aspects of the present invention, the comparison module, or any other module of the invention, can include an operating system (e.g., UNIX, Windows) on which runs a relational database management system, a World Wide Web application, and a World Wide Web server. World Wide Web application can includes the executable code necessary for generation of database language statements [e.g., Standard Query Language (SQL) statements]. The executables can include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using an HTML interface provided by Web browsers and Web servers. In other embodiments of the invention, other interfaces, such as HTTP, FTP, SSH and VPN based interfaces can be used to connect to the Internet databases.

In some embodiments of this aspect and all other aspects of the present invention, a computer-readable media can be transportable such that the instructions stored thereon, such as computer programs and software, can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement aspects of the present invention. The computer executable instructions can be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The computer instructions can be implemented in software, firmware or hardware and include any type of programmed step undertaken by modules of the information processing system. The computer system can be connected to a local area network (LAN) or a wide area network (WAN). One example of the local area network can be a corporate computing network, including access to the Internet, to which computers and computing devices comprising the data processing system are connected. In one embodiment, the LAN uses the industry standard Transmission Control Protocol/Internet Protocol (TCP/IP) network protocols for communication. Transmission Control Protocol Transmission Control Protocol (TCP) can be used as a transport layer protocol to provide a reliable, connection-oriented, transport layer link among computer systems. The network layer provides services to the transport layer. Using a two-way handshaking scheme, TCP provides the mechanism for establishing, maintaining, and terminating logical connections among computer systems. TCP transport layer uses IP as its network layer protocol. Additionally, TCP provides protocol ports to distinguish multiple programs executing on a single device by including the destination and source port number with each message. TCP performs functions such as transmission of byte streams, data flow definitions, data acknowledgments, lost or corrupt data re-transmissions, and multiplexing multiple connections through a single network connection. Finally, TCP is responsible for encapsulating information into a datagram structure. In alternative embodiments, the LAN can conform to other network standards, including, but not limited to, the International Standards Organization's Open Systems Interconnection, IBM's SNA, Novell's Netware, and Banyan VINES.

In some embodiments, the computer system as described herein can include any type of electronically connected group of computers including, for instance, the following networks: Internet, Intranet, Local Area Networks (LAN) or Wide Area Networks (WAN). In addition, the connectivity to the network may be, for example, remote modem, Ethernet (IEEE 802.3), Token Ring (IEEE 802.5), Fiber Distributed Datalink Interface (FDDI) or Asynchronous Transfer Mode (ATM). The computing devices can be desktop devices, servers, portable computers, hand-held computing devices, smart phones, set-top devices, or any other desired type or configuration. As used herein, a network includes one or more of the following, including a public internet, a private internet, a secure internet, a private network, a public network, a value-added network, an intranet, an extranet and combinations of the foregoing.

In one embodiment of the invention, the computer system can comprise a pattern comparison software can be used to determine whether the patterns of data of the amount of cells of test cancer cells on a receiving substrate and/or on a seed-substrate are indicative of that cancer population having a high invasive capacity and comprising a metastatic cancer population, and predictive of a metastatic cancer in a subject. In this embodiment, the pattern comparison software can compare at least some of the data (e.g., data of the amount of cells of test cancer cells on a receiving substrate and/or on a seed-substrate) with reference data (e.g., data of the amount of a high-metastatic cell population, or a low-metastatic cell population) to determine how closely they match. The matching can be evaluated and reported in portions or degrees indicating the extent to which all or some of the pattern matches.

In some embodiments of this aspect and all other aspects of the present invention, a comparison module provides computer readable data that can be processed in computer readable form by predefined criteria, or criteria defined by a user, to provide a retrieved content that may be stored and output as requested by a user using a display module.

Output Module

In accordance with some embodiments of the invention, the computerized system can include or be operatively connected to an output module. In some embodiments, the output module is a display module, such as computer monitor, touch screen or video display system. The display module allows user instructions to be presented to the user of the system, to view inputs to the system and for the system to display the results to the user as part of a user interface. Optionally, the computerized system can include or be operative connected to a printing device for producing printed copies of information output by the system.

In some embodiments, the results can be displayed on a display module or printed in a report, e.g., a to indicate the invasive potential and/or metastatic potential of a population of cancer cells of interest, e.g., a low, medium or high risk of likelihood of the cancer cell population comprising metastatic cancer cells.

In some embodiments, the report is a hard copy printed from a printer. In alternative embodiments, the computerized system can use light or sound to report the result, e.g., to indicate the invasive capacity and/or metastatic potential of the cancer cell population. For example, in all aspects of the invention, the report produced by the methods, assays, systems and kits as disclosed herein can comprise a report which is color coded to signal or indicate the invasive potential and/or metastatic potential of a population of cancer cells of interest, e.g., a low, medium or high risk of likelihood of the cancer cell population comprising metastatic cancer cells, or compared another “gold” standard highly-metastatic or low-metastatic reference control cell line of the investigators choice.

For example, a red color or other predefined signal can indicate that the test cancer cell population has a similar invasive capacity as a highly-metastatic positive control line, thus signaling that the cancer cell population comprises highly-metastatic cancer cells. In another embodiment, a yellow or orange color or other predefined signal can indicate that the test cancer cell population has a similar invasive capacity as a low-metastatic positive control line, thus signaling that the cancer cell population comprises low-metastatic cancer cells. In another embodiment, a green color or other predefined signal can indicate that the test cancer cell population has a does not have an invasive capacity (e.g., is similar to a non-metastatic negative reference control line), thus signaling that the cancer cell population likely does not comprise metastatic cancer cells. In some embodiments, a “heat map” or gradient color scheme can be used in the report to signal the metastatic potential and/or invasive capacity of the test cancer cell population, where fir example, the gradient is a red to yellow to green gradient, where a red signal will signal an cancer cell line with high metastatic potential and high invasive capacity, and a yellow signal will indicate a cancer cell line with low metastatic potential and low invasive capacity and a green signal will indicate a cancer cell line with negligible to metastatic potential or invasive capacity. Typically, a gradient, whether using arbitrary numerical scale or a color gradient is based upon one or more reference cancer cell lines, for example, a red scale would be established by highly-metastatic positive reference cell lines, and yellow by low-metastatic positive cell lines, and green by negative control cancer cell lines. Colors between red and yellow and yellow and green will signal the characteristics of the invasiveness and propensity to be a metastatic cancer cell line with respect to a red-yellow-green scale. Other color schemes and gradient schemes in the report are also encompassed.

In some embodiments, the report can display the % migration (as compared to the starting population), and/or absolute amount of cells on the receiving substrate, or a numerical number grade or % value as compared with a reference positive cancer cell line or a negative reference cancer cell line.

In some embodiments, the report can display the normalized values of the test cancer cell population, which are normalized to a reference positive highly-metastatic and/or positive low-metastatic cancer cell line (e.g., a selected “gold” standard highly-metastatic cell line of the investigators choice). Accordingly, a report can display the % difference, and/or the change in absolute number of amount of cells on the receiving substrate as compared to the absolute amount of cells of the positive reference (highly-metastatic and/or positive low-metastatic) cancer cell line. Similarly, the report can display the % difference as compared to the amount of cells of a positive or negative reference cancer cell line. As an illustrative example only, the report can indicate that the test cancer cell population has a 34% increase of amount of cells as compared to a negative control cell line. Alternatively, the report can indicate that the test cancer cell population has a 70% decrease in the amount of cells as compared to a positive highly-metastatic cancer cell line.

In some embodiments, the report can be color-coded, for instance, if the % or absolute number of cells on the receiving substrate of the test cancer cell sample is above a certain pre-defined threshold level, the color of the % value or absolute number of cells can be a bright color (e.g., red), or otherwise marked (e.g. by a *) or highlighted for easy identification that this value indicates that the cancer cell population comprises a population of metastatic cancer cells and has a high invasive capacity. The pre-defined threshold level is determined by the investigator, and is typically determined by a comparison of the values for the % and absolute number of cells of reference positive cancer cell lines with known metastatic potential and invasive capacity. In some embodiments the threshold is at least about 10%, or at least 20%, at least 33%, or at least 40% or at least 45% or greater than 45% of a negative reference cancer cell line, for example where the amount of cells on a receiving substrate is at least 35%, or at least 40% or at least 45% or above 45% above the amount of cells on receiving substrate of a negative reference cell lines, it is indicative that the cancer cell population comprises a population of metastatic cancer cells and has a high invasive capacity.

In some embodiments, the report can also display the reference values (either in % or absolute amount of cells on the receiving substrate) of reference cancer cell line. In some embodiments, reference values for at least one positive reference cancer line are shown, e.g., a highly-metastatic and/or low-metastatic reference cancer cell line. In some embodiments, reference values for at least one negative reference cancer line is shown, e.g., a non-metastatic a reference cancer cell line. Such reference values can be used to compare with the values from the test cancer cell population.

In some embodiments, the report can also present text, either verbally or written, giving a recommendation of the invasive capacity and/or metastatic potential of the cancer cell population. In other embodiments, the report provides just values or numerical scores for the invasive capacity or metastatic potential which can be readily compared by a physician with reference values as disclosed herein.

In some embodiments of this aspect and all other aspects of the present invention, the report data from the comparison module can be displayed on a computer monitor as one or more pages of the printed report. In one embodiment of the invention, a page of the retrieved content can be displayed through printable media. The display module can be any device or system adapted for display of computer readable information to a user. The display module can include speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc

In some embodiments of the present invention, a World Wide Web browser can be used to provide a user interface to allow the user to interact with the system to input information, construct requests and to display retrieved content. In addition, the various functional modules of the system can be adapted to use a web browser to provide a user interface. Using a Web browser, a user can construct requests for retrieving data from data sources, such as data bases and interact with the comparison module to perform comparisons and pattern matching. The user can point to and click on user interface elements such as buttons, pull down menus, scroll bars, etc. conventionally employed in graphical user interfaces to interact with the system and cause the system to perform the methods of the invention. The requests formulated with the user's Web browser can be transmitted over a network to a Web application that can process or format the request to produce a query of one or more database that can be employed to provide the pertinent information related to the tumor type, the retrieved content, process this information and output the results, e.g. at least one of any of the following: % invasion, % invasion under specific conditions (e.g., culture time, presence of drugs, tumor of different geometry, e.g., with or without hypoxic cells). In some embodiments, these values or their combination can exhibit strong correlation with invasive capacity of cells in the patients. In some embodiments, output information of the % invasion, % invasion under specific conditions (e.g., culture time, presence of drugs, tumor of different geometry, e.g., with or without hypoxic cells) can vary with different tumor types, and can be determined by one of ordinary skill in the art by comparing the numbers across a range of highly metastatic cancer cell lines as disclosed herein.

### Cancer Cell Types

In some embodiments, the biological sample comprising cancer cells can be a single cell or homogenized cancer biopsy sample. In some embodiments, the biological sample comprises cancer cells which have been cultured in vitro. In alternative embodiments, the biological sample is a portion of a biopsy sample obtained from the subject, and can be placed directly onto the hydrogel in the seed-substrate to assess cancer cells present in solid tumors.

In some embodiments, a biological sample comprising cancer cells is serum plasma, blood or tissue sample, for example, wherein the biological sample is selected from the group consisting of; a tissue sample; a tumor sample; a tumor cell; a biopsy sample; ex vivo cultivated sample; ex vivo cultivated tumor sample; surgically dissected tissue sample, blood sample, plasma sample, cancer sample, lymph fluid sample or primary ascite sample. In alternative embodiments, the biological sample includes, for example blood, plasma, serum, urine, spinal fluid, plural fluid, nipple aspirates, lymph fluid, external secretions of the skin, respiratory, internal and genitourinary tracts, bile, tears, sweat, saliva, organs, milk cells and primary ascite cells, biopsy tissue sample, a cancer biopsy tissue sample, an in vitro or ex vivo cultivated biopsy tissue sample.

The 3-dimensional invasive assay as disclosed herein can be used to assess the invasive potential of any desired cancer cell type. Cancers include, but are not limited to, bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS associated leukemias and adult T-cell leukemia lymphoma; intraepithelial neoplasms including Bowen's disease and Paget's disease, liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilm's tumor.

In some embodiments, a three dimensional invasive assay as disclosed herein can be used to profile multiple samples isolated from patients with prostate cancer (e.g., tumor samples from prostatectomy), as well as breast cancer cells (e.g., tumor samples from mastectomies), as well as brain and bone cancers.

In one embodiment, the subject is assessed if they are at risk of having a metastasis or malignant cancer, the method comprising assessing an invasive potential of a cancer from the subject using the 3D cellular invasive assay as disclosed herein, and if the level of cells measured on a receiving substrate are above a pre-determined reference threshold level, the subject is at risk of having a metastasis or a malignant cancer. In some embodiments, the reference threshold level the based on the level of migration of a non-metastatic cancer cell or a control cell line (e.g., fibroblast cells or prostate epithelial cells (RWPE-1), or cells from a normal tissue sample, where in the tissue sample is a biological tissue sample from a tissue matched, and species matched and age matched biological sample. In some embodiments, the reference level is based on a reference sample is from a non-malignant matched tissue sample. In some embodiments, the reference level is based on a reference sample from a non-malignant cancer tissue sample.

### Kits

Another aspect of the present invention relates to a kit for carrying out a method as disclosed herein, where the kit comprises: (i) at least one porous, hydrophilic seed-substrate comprising a plurality of defined regions comprising hydrogels disposed within; (ii) at least one porous, hydrophilic receiving-substrate comprising a plurality of defined regions comprising hydrogels disposed within; and (iii) at least reference control tumor cell line.

In some embodiments, the seed-substrate comprises a reference control tumor cell line is disposed within at least one hydrogel location of the seed-substrate, thus serving as an internal control. In some embodiments, a reference control tumor cell line is a positive reference cancer cell line, e.g., a highly metastatic tumor cell, or a low-metastatic cancer cell line, for example, such as those as disclosed herein. In some embodiments, the seed-substrate comprises can also comprise a reference control tumor cell line which is disposed within at least one hydrogel location of the seed-substrate, which is a negative control, e.g., a non-metastatic tumor cell line. In alternative embodiments, a plurality of hydrogels in the seed-substrate can be spiked with an inhibitor of actin mobility, which where the hydrogel is also seeded with a biological sample comprising cancer cells of interest, such a well can serve as an internal negative control for a direct comparison with the cancer cells in the biological sample of interest.

In some embodiments, the kits of as disclosed herein can comprise culture media. In some embodiments, the kit can comprise receiving substrates which comprise additional cells, e.g., EMC cells in the hydrogel locations on the receiving substrates.

In some embodiments, the kit can further comprise instructions for assembly of the three-dimensional in vitro invasion assay as disclosed herein, e.g., instructions on the predefined pre-incubation of the seed-substrate prior to stacking, instructions for the predefined period of incubation for the migration to occur, as well as instructions how to stack the seed-substrates with at least one receiving substrates. The kit can also optionally provide instructions on optimal numbers of seed substrates, and optimal numbers of receiving substrates etc to use in the 3-dimensional in vitro invasive assay, as well as suitable configurations, and combinations of stacks cell-containing receiving layers with cells, or “blank” receiving layers.

In some embodiments, a kit as disclosed herein also comprises at least one reagent for measuring the amount of cells on the substrates after separation of the seed-substrate from one or more receiving substrates. Such agents are well known in the art, and include without limitation, labeled antibodies to select for cancer cell markers and the like. In some embodiments, the labeled antibodies are fluorescently labeled, or labeled with magnetic beads and the like. In some embodiments, a kit as disclosed herein can further comprise at least one or more reagents for profiling and annotating a metastatic cancer cell population in high throughput, etc. according to the methods as disclosed herein.

In addition to the above mentioned component(s), the kit can also include informational material. The informational material can be descriptive, instructional, marketing or other material that relates to the methods described herein and/or the use of the components for the assays, methods and systems described herein. For example, the informational material may describe methods for enriching the metastatic cancer cell population, for characterizing a plurality of properties of a metastatic cancer cell population.

In some embodiments, the methods, systems, kits and devices as disclosed herein can be performed by a service provider, for example, where an investigator can have one or more samples (e.g., an array of samples) each sample comprising a population of cancer cells, for assessment using the methods, kits and systems as disclosed herein in a diagnostic laboratory operated by the service provider. In such an embodiment, after performing the assays, methods and systems of the invention as disclosed, the service provider can performs the analysis and provide the investigator a report as disclosed herein of the characteristics of each cancer cell population analyzed.

In alternative embodiments, a service provider can provide the investigator with the raw data of the assays and leave the analysis to be performed by the investigator. In some embodiments, the report is communicated or sent to the investigator via electronic means, e.g., uploaded on a secure web-site, or sent via e-mail or other electronic communication means. In some embodiments, the investigator can send the samples to the service provider via any means, e.g., via mail, express mail, etc., or alternatively, the service provider can provide a service to collect the samples from the investigator and transport them to the diagnostic laboratories of the service provider. In some embodiments, the investigator can deposit the samples to be analyzed at the location of the service provider diagnostic laboratories. In alternative embodiments, the service provider provides a stop-by service, where the service provider send personnel to the laboratories of the investigator and also provides the kits, apparatus, and reagents for performing the assays, methods and systems of the invention as disclosed herein of the investigators of the cancer cell populations in the investigators laboratories, and analyses the result and provides a report to the investigator of the characteristics of each cancer cell population, or a plurality of cancer cell populations analyzed.

### Uses of the Three-Dimensional In Vitro Invasive Assay

Another aspect of the invention is use of the three-dimensional in vitro invasion assay in a method for identifying an agent that inhibits a metastatic cell, comprising measuring the amount of cells on a receiving substrate after a predefined period of time in the presence and absence of a putative compound or agent; wherein the amount of cells on a receiving substrate is proportional to (a function of) the metastatic potential (ability, activity, capacity) of the cell; and wherein a significant lower amount of cells on the receiving substrate in the presence of an agent, as compared to the absence of such an agent is indicates that the putative agent is effective to inhibit cancer metastasis. The amount of cells on the receiving substrate can be determined by measuring the quantity of cells according to any method as disclosed herein, or the amount of the detectable marker (e.g., a fluorescent marker or luciferase) as disclosed herein. In some embodiments, the amount of inhibition of the agent can be compared to a positive control agent, e.g., an agent which is a specific inhibitor of actin-based mobility, such as cytoclastin D as disclosed herein (FIG. 1).

Another embodiment relates to use of a three-dimensional in vitro invasion assay as disclosed herein in a method to enrich for a population of metastatic cancer cells. In some embodiments, this encompasses enriching a population of metastatic cancer cells from a mixed population of metastatic and non-metastatic cancer cells or non-cancer cells. As used herein, the term “enriching” or “enrich for” are used interchangeably, and refers to increasing the population of cells of interest, for example ovarian cancer stem cells in a population of cells, for example increasing the percentage of ovarian cancer stem cells by about 10% or about 20% or about 30%, or about 40% or about 50% or about 60% or greater than 60% within the total population cells as compared to the starting population of cells. In some embodiments, a purified or substantially pure population of metastatic cancer cells can be used for genomic analysis by techniques such as microarray hybridization, SAGE, MPSS, or proteomic analysis to identify more markers that characterize the metastatic cancer cell populations which can be used in methods to distinguish them from non-metastatic cancer cell populations.

Also encompassed in the present invention is the use of the three-dimensional in vitro invasion assay as disclosed herein in a method to identify agents which kill and/or decrease the rate of migration or invasion of metastatic cancer cells into surrounding tissues. In some embodiments, such a three-dimensional in vitro invasion assay as disclosed herein can be used to enrich for metastatic cancer cells and then be used in an assay which selectively targets inhibition of proliferation or death of a metastatic cancer cell population. Once can measure and compare the rate of proliferation of a metastatic cancer cell population enriched using the methods as disclosed herein with a non-metastatic cancer cell population using the methods for example the MTT assay or CFU assay, and an agent identified to decrease the rate of proliferation and/or attenuate proliferation by about 10%, or about 20% or about 30% or greater than 30% and/or kill about 10% or about 20% or about 30% or greater than 30% of the population of metastatic cancer cell population as compared to a population of non-metastatic cancer cells identifies an agent that is useful for a therapy for a metastatic cancer. Effectively, the assay as disclosed herein can be used to identify agents that selectively inhibit a metastatic cancer cell population as compared to a non-metastatic cancer cell population. Agents useful in such an embodiment can be any agent as disclosed herein under the term “agent” in the definitions section, and can be for example nucleic acid agents, such as RNAi agents (RNA interference agents), nucleic acid analogues, small molecules, proteins, peptidomimetics, antibodies, peptides, aptamers, ribozymes, and variants, analogues and fragments thereof.

In further embodiments, a three-dimensional in vitro invasion assay as disclosed herein can be used in a method to identify and/or enrich for specific metastatic cancer cell populations in different cancer types and populations of cancer cells, and can be enriched and used for the study and understanding of signaling pathways of metastatic cancer cells. The isolation of different metastatic cancer cell populations is useful to aid the development of therapeutic applications for metastatic cancers.

Additionally, a three-dimensional in vitro invasion assay as disclosed herein can be used in a method to identify additional markers on metastatic cancer cells that characterize them as metastatic cells as compared to non-metastatic cells. Such markers can be cell-surface markers or surface markers or other markers, for example mRNA or protein markers intracellular within the cell. Such markers can be used in the development of as additional agents and tools for additional diagnosis of metastatic cancer cells, which can be used for in vitro, ex vivo or in vivo diagnostic assay applications to identify metastatic cancers in subjects with cancers.

In further embodiments, the metastatic cancer cells which can be identified and isolated using a three-dimensional in vitro invasion assay as disclosed herein can be used in a method to prepare antibodies that are specific to markers of metastatic cancers cells. Polyclonal antibodies can be prepared by injecting a vertebrate animal with cells of this invention in an immunogenic form. Production of monoclonal antibodies is described in such standard references as U.S. Pat. Nos. 4,491,632, 4,472,500 and 4,444,887, and Methods in Enzymology 73B:3 (1981). Specific antibody molecules can also be produced by contacting a library of immunocompetent cells or viral particles with the target antigen, and growing out positively selected clones. See Marks et al., New Eng. J. Med. 335:730, 1996, and McGuiness et al., Nature Biotechnol. 14:1449, 1996. A further alternative is reassembly of random DNA fragments into antibody encoding regions, as described in EP patent application 1,094,108 A.

The antibodies in turn can be used as diagnostic applications to identify a subject with a cancer comprising metastatic cancers cells, or alternatively, antibodies can be used as therapeutic agents to prevent the migration and intravasation of metastatic cancers cells in the subject.

In another embodiment, three-dimensional in vitro invasion assay as disclosed herein can be used in a method to enrich for a metastatic cancers cell population which can be used to prepare a cDNA library of relatively uncontaminated with cDNAs that are preferentially expressed in metastatic cancer cells of a certain cancer type as compared to non-metastatic cancer cells of the same cancer type. For example, metastatic cancer cells are collected and then mRNA is prepared from the pellet by standard techniques (Sambrook et al., supra). After reverse transcribing the cDNA, the preparation can be subtracted with cDNA from, for example non-stem cell ovarian cancer cells or non-cancer ovarian cells in a subtraction cDNA library procedure. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the molecular size or amount of mRNA transcripts between two samples.

Any suitable method for detecting and comparing mRNA expression levels in a sample can be used in connection with the methods of the invention. For example, mRNA expression levels in a sample can be determined by generation of a library of expressed sequence tags (ESTs) from a sample. Enumeration of the relative representation of ESTs within the library can be used to approximate the relative representation of a gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript. The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680. Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505. Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined. Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes. Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992. General methods in molecular and cellular biochemistry can also be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad™, Stratagene™, Invitrogen™, Sigma-Aldrich™, and ClonTech™.

### Administration of Pharmaceutical Compositions

Another aspect of the present invention relates to a method of treating a subject identified as having a cancer with a metastatic potential and invasive capacity as determined by the method disclosed herein using the three-dimensional in vitro invasive assay. In some embodiments, a subject identified to be at risk for having a cancer with metastatic potential and/or invasive capacity can be administered an appropriate anti-cancer agent. Where a subject is identified to comprise a cancer with a highly-metastatic cancer, a physician can recommend an aggressive regimen for the treatment of cancer. Such aggressive regimes are well known by ordinary physicians in the art of oncology and cancer treatment.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

After formulation with an appropriate pharmaceutically acceptable carrier in a desired dosage, the pharmaceutical compositions of this invention can be administered to a subject. The pharmaceutical compositions of this invention can be administered to a subject using any suitable means. In general, suitable means of administration include, but are not limited to, topical, oral, parenteral (e.g., intravenous, subcutaneous or intramuscular), rectal, intracisternal, intravaginal, intraperitoneal, ocular, or nasal routes.

Cancer therapy can also include prophylaxis, including agents which slow or reduce the risk of cancer in a subject. In other embodiments, a cancer therapy is any treatment or any means to prevent the proliferation of cells with abnormal proliferation or cancerous cells. In some embodiments, then anti-cancer treatment is an agent which suppresses the EGF-EGFR pathway, for example but not limited to inhibitors and agents of EGFR Inhibitors of EGFR include, but are not limited to, tyrosine kinase inhibitors such as quinazolines, such as PID 153035, 4-(3-chloroanilino) quinazoline, or CP-358,774, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d]pyrimidines (Traxler et al., (1996) J. Med Chem 39:2285-2292), curcumin (diferuloyl methane) (Laxmin arayana, et al., (1995), Carcinogen 16:1741-1745), 4,5-bis(4-fluoroanilino) phthalimide (Buchdunger et al. (1995) Clin. Cancer Res. 1:813-821; Dinney et al. (1997) Clin. Cancer Res. 3:161-168); tyrphostins containing nitrothiophene moieties (Brunton et al. (1996) Anti Cancer Drug Design 11:265-295); the protein kinase inhibitor ZD-1 839 (AstraZeneca); CP-358774 (Pfizer, Inc.); PD-01 83805 (Warner-Lambert), EKB-569 (Torrance et al., Nature Medicine, Vol. 6, No. 9, September 2000, p. 1024), HKI-272 and HKI-357 (Wyeth); or as described in International patent application WO05/018677 (Wyeth); WO99/09016 (American Cyanamid); WO98/43960 (American Cyanamid); WO 98/14451; WO 98/02434; WO97/38983 (Warener Labert); WO99/06378 (Warner Lambert); WO99/06396 (Warner Lambert); WO96/30347 (Pfizer, Inc.); WO96/33978 (Zeneca); WO96/33977 (Zeneca); and WO96/33980 (Zeneca), WO 95/19970; U.S. Pat. App. Nos. 2005/0101618 assigned to Pfizer, 2005/0101617, 20050090500 assigned to OSI Pharmaceuticals, Inc.; all herein incorporated by reference. Further useful EGFR inhibitors are described in U.S. Pat. App. No. 20040127470, particularly in tables 10, 11, and 12, and are herein incorporated by reference.

In another embodiment, the anti-cancer therapy includes a chemotherapeutic regimen further comprises radiation therapy. In an alternate embodiment, the therapy comprises administration of an anti-EGFR antibody or biological equivalent thereof.

In some embodiments, the anti-cancer treatment comprises the administration of a chemotherapeutic drug selected from the group consisting of fluoropyrimidine (e.g., 5-FU), oxaliplatin, CPT-11, (e.g., irinotecan) a platinum drug or an anti EGFR antibody, such as the cetuximab antibody or a combination of such therapies, alone or in combination with surgical resection of the tumor. In yet a further aspect, the treatment compresses radiation therapy and/or surgical resection of the tumor masses. In one embodiment, the present invention encompasses administering to a subject identified as having, or increased risk of developing RCC an anti-cancer combination therapy where combinations of anti-cancer agents are used, such as for example Taxol, cyclophosphamide, cisplatin, gancyclovir and the like. Anti-cancer therapies are well known in the art and are encompassed for use in the methods of the present invention. Chemotherapy includes, but is not limited to an alkylating agent, mitotic inhibitor, antibiotic, or antimetabolite, anti-angiogenic agents etc. The chemotherapy can comprise administration of CPT-11, temozolomide, or a platin compound. Radiotherapy can include, for example, x-ray irradiation, w- irradiation, γ-irradiation, or microwaves.

The term “chemotherapeutic agent” or “chemotherapy agent” are used interchangeably herein and refers to an agent that can be used in the treatment of cancers and neoplasms, for example brain cancers and gliomas and that is capable of treating such a disorder. In some embodiments, a chemotherapeutic agent can be in the form of a prodrug which can be activated to a cytotoxic form. Chemotherapeutic agents are commonly known by persons of ordinary skill in the art and are encompassed for use in the present invention. For example, chemotherapeutic drugs for the treatment of tumors and gliomas include, but are not limited to: temozolomide (Temodar), procarbazine (Matulane), and lomustine (CCNU). Chemotherapy given intravenously (by IV, via needle inserted into a vein) includes vincristine (Oncovin or Vincasar PFS), cisplatin (Platinol), carmustine (BCNU, BiCNU), and carboplatin (Paraplatin), Mexotrexate (Rheumatrex or Trexall), irinotecan (CPT-11); erlotinib; oxalipatin; anthracyclins- idarubicin and daunorubicin; doxorubicin; alkylating agents such as melphalan and chlorambucil; cis-platinum, methotrexate, and alkaloids such as vindesine and vinblastine.

Some examples of anti-VEGF agents include bevacizumab (Avastin™), VEGF Trap, CP-547,632, AG13736, AG28262, SU5416, SU11248, SU6668, ZD-6474, ZD4190, CEP-7055, PKC 412, AEE788, AZD-2171, sorafenib, vatalanib, pegaptanib octasodium, IM862, DC101, angiozyme, Sirna-027, caplostatin, neovastat, ranibizumab, thalidomide, and AGA-1470, a synthetic analog of fumagillin (alternate names: Amebacilin, Fugillin, Fumadil B, Fumadil) (A. G. Scientific, catalog #F1028), an angio-inhibitory compound secreted by Aspergillus fumigates.

As used herein the term “anti-VEGF agent” refers to any compound or agent that produces a direct effect on the signaling pathways that promote growth, proliferation and survival of a cell by inhibiting the function of the VEGF protein, including inhibiting the function of VEGF receptor proteins. The term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies. Preferred VEGF inhibitors, include for example, AVASTIN® (bevacizumab), an anti-VEGF monoclonal antibody of Genentech, Inc. of South San Francisco, Calif., VEGF Trap (Regeneron/Aventis). Additional VEGF inhibitors include CP-547,632 (3-(4-Bromo-2,6-difluoro- benzyloxy)-5-[3-(4-pyrrolidin 1-yl- butyl)-ureido]-isothiazole-4- carboxylic acid amide hydrochloride; Pfizer Inc., NY), AG13736, AG28262 (Pfizer Inc.), SU5416, SU11248, & SU6668 (formerly Sugen Inc., now Pfizer, New York, N.Y.), ZD-6474 (AstraZeneca), ZD4190 which inhibits VEGF-R2 and —R1 (AstraZeneca), CEP-7055 (Cephalon Inc., Frazer, Pa.), PKC 412 (Novartis), AEE788 (Novartis), AZD-2171), NEXAVAR® (BAY 43-9006, sorafenib; Bayer Pharmaceuticals and Onyx Pharmaceuticals), vatalanib (also known as PTK-787, ZK-222584: Novartis & Schering: AG), MACUGEN® (pegaptanib octasodium, NX-1838, EYE-001, Pfizer Inc./Gilead/Eyetech), IM862 (glufanide disodium, Cytran Inc. of Kirkland, Wash., USA), VEGFR2-selective monoclonal antibody DC101 (ImClone Systems, Inc.), angiozyme, a synthetic ribozyme from Ribozyme (Boulder, Colo.) and Chiron (Emeryville, Calif.), Sirna-027 (an siRNA-based VEGFR1 inhibitor, Sirna Therapeutics, San Francisco, Calif.) Caplostatin, soluble ectodomains of the VEGF receptors, Neovastat VEterna Zentaris Inc; Quebec City, Calif.) and combinations thereof.

The compositions as disclosed herein used in connection with the treatment methods of the present invention are administered and dosed in accordance with good medical practice, taking into account the clinical condition of the individual subject, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

The present invention may be as defined in any one or more of the following numbered paragraphs.
1. A method for assessing the metastatic potential of a population of cancer cells, the method comprising:
   a. contacting a biological sample comprising a population of cancer cells at a defined region of a seed-substrate comprising a hydrogel, wherein the seed-substrate is a porous, hydrophilic substrate layer of a three-dimensional cellular assay, wherein the seed-substrate is in contact with at least one receiving-substrate to form a multi-layered three-dimensional cellular assay, wherein the receiving-substrate is a porous, hydrophilic substrate comprising a hydrogel at defined regions;
   b. separating the seed-substrate and the at least one receiving-substrate of the three-dimensional cellular assay after incubation of the three-dimensional cellular assay for a predefined period of time;
   c. measuring the quantity of cancer cells on the, at least one, receiving-substrate, where the amount of cancer cells on the receiving-substrate is indicative of the metastatic potential of the population of cancer cells.
2. The method of paragraph 1, further comprising measuring the quantity of cancer cells on at least the seed-substrate layer. In such an embodiment, few cells remaining on the seed-substrate layer indicate that most cells have moved. In alternative embodiment, many cells on the seed-substrate layer could indicate that no cells have moved, or that cells have proliferated and moved, or moved and then proliferated. In some embodiments, the assay comprises a control seed-substrate layer with no receiving layers, where analysis of the viability and/or proliferation of the cells after the predetermined time of incubation in the assay is performed to determine the viability of the cells and the proliferation rate of the cells. In some embodiments, if no cells move, the cells on the seed-substrate could be non-metastatic or could be non-viable.
3. The method of paragraph claim 1, wherein the seed-substrate is in contact with at least one lower-receiving substrate.
4. The method of paragraph 1, wherein the seed-substrate is in contact with at least one upper-receiving substrate.
5. The method of claim 1, wherein the seed-substrate is in contact with at least one upper-receiving substrate and at least one lower substrate.
6. The method of paragraph 1, wherein the three-dimensional cellular assay comprises a plurality of receiving substrates, wherein the each receiving substrate is layered on at least one other receiving substrate.
7. The paragraph of claim 6, wherein the three-dimensional cellular assay comprises between 1 and 50 receiving substrates.
8. The method of paragraph 6, wherein the three-dimensional cellular assay comprises between 50 and 100 receiving substrates.
9. The method of paragraph 1, wherein the at least one receiving substrate can comprise extracellular matrix (ECM) cells.
10. The method of paragraph 1, wherein the seed-substrate and the at least one receiving-substrate comprises a matrix with a pore-diameter to allow invasion of metastatic cells.
11. The method of paragraph 1, wherein the porous, hydrophilic substrate of the seed-substrate and the at least one receiving-substrate is paper.
12. The method of paragraph 1, wherein the porous, hydrophilic substrate of the seed-substrate and the at least one receiving-substrate is acellulose-based substrate, a polymeric substrate, a, nitrocellulose substrate or a cellulose acetate substrate or a combination thereof, wherein each substrate has a pre-defined pore size.
13. The method of paragraph 1, further comprising measuring the quantity of cancer cells on the seed-substrate, wherein a high percentage of cancer cells on the seed-substrate as compared to the percentage of cells on the receiving substrate is indicative of a low metastatic potential of the population of cancer cells.
14. The method of paragraph 1, further comprising comparing the measured quantity of cancer cells on the seed-substrate with the measured quantity of cancer cells on at least one receiving-substrate at a predefined timepoint.
15. The method of paragraph 1, wherein the biological sample comprises a predefined number of cancer or tumor cells.
16. The method of paragraph 15, further comprising comparing the predefined number of cancer cells in a biological sample with the measured quantity of cancer cells on the receiving substrate at a predefined timepoint.
17. The method of paragraph 15, further comprising comparing the predefined number of cancer cells in a biological sample with the measured quantity of cancer cells on the seed-substrate at a predefined timepoint.
18. The method of paragraph 1, wherein the biological sample comprises a portion of a cancer biopsy sample.
19. The method of paragraph 1, wherein the biological sample comprises a cell suspension of cancer cells.
20. The method of paragraph 17, wherein the cancer cells are obtained from a tumor biopsy obtained from a subject.
21. The method of paragraph 1, further comprising labeling the cancer cells in the biological sample prior to contacting with the seed-substrate.
22. The method of paragraph 1, further comprising labeling the cancer cells within the three-dimensional cellular assay prior to, or after separation of the seed-substrate with the at least one receiving substrate.
23. The method of paragraph 1, further comprising fixing the cancer cells in the three-dimensional cellular assay prior to, or after separation of the seed-substrate with the at least one receiving substrate.
24. The method of paragraph 1, wherein the measuring the quantity of the cancer cells is by image analysis.
25. The method of paragraph 1, wherein the measuring the quantity of the cancer cells can be by any or a combination of the following: cell proliferation assays, fluorescent labeling assays, standard cytometry assays, heamocytometer, automated cell counter, and flow cytometer.
26. The method of paragraph 1, wherein measuring the quantity of the cancer cells is performed after the cancer cells are removed from the seed-substrate or at least one receiving substrate.
27. The method of paragraph 1, wherein measuring the quantity of the cancer cells is performed by measuring gene expression or protein expression of predetermined selected genes.
28. The method of paragraph 9, wherein at least one receiving substrate comprises an additional cell in the hydrogel.
29. The method of paragraph 28, wherein the additional cells are selected from the group of endothelial cells, stromal cells, fibroblast cells, or any combination thereof
30. An assay comprising:
   a. a porous, hydrophilic seed-substrate comprising a plurality of defined regions comprising hydrogels disposed within a seed-substrate, wherein at least one hydrogel comprises a cancer cell population, and at least one hydrogel comprises a reference control cancer cell line;
   b. at least one porous, hydrophilic receiving-substrate comprising a plurality of defined regions comprising hydrogels; wherein the receiving substrate is stacked above or below the seed-substrate.
31. The assay of paragraph 30, wherein the reference control cell line is positive control line which is a highly-metastatic cancer cell line.
32. The assay of paragraph 30, wherein the reference control cell line is positive control line which is a low-metastatic cancer cell line.
33. The assay of paragraph 30, wherein the reference control cell line is a non-metastatic cancer cell line.
34. The assay of paragraph 30, wherein the reference control cell line is negative control line
35. The assay of paragraph 30, wherein the hydrogels in the at least one receiving substrate can comprise additional cells.
36. The method of paragraph 35, wherein the additional cells are endothelial cells, stromal cells, fibroblast cells, or any combination thereof
37. A method to identify if a subject has a metastatic cancer, the method comprising:
   a. contacting a biological sample comprising a population of cancer cells obtained from the subject at a defined region of a seed-substrate comprising a hydrogel, wherein the seed-substrate is a porous, hydrophilic substrate layer of a three-dimensional cellular assay, wherein the seed-substrate is in contact with at least one receiving-substrate to form a multi-layered three-dimensional cellular assay, wherein the receiving-substrate is a porous, hydrophilic substrate comprising a hydrogel at defined regions,
   b. separating the seed-substrate and the at least one receiving-substrate of the three-dimensional cellular assay after incubation of the three-dimensional cellular assay for a predefined period of time;
   c. measuring the quantity of cancer cells on the at least one receiving-substrate, where the amount of cancer cells on the receiving-substrate is indicative of the subject having a metastatic cancer.
38. A kit comprising:
   a. at least one porous, hydrophilic seed-substrate comprising a plurality of defined regions comprising hydrogels disposed within;
   b. at least one porous, hydrophilic receiving-substrate comprising a plurality of defined regions comprising hydrogels disposed within; and
   c. at least reference control tumor cell line.
39. The kit of paragraph 38, wherein the reference control tumor cell line is disposed within at least one hydrogel location of the seed-substrate.
40. The kit of paragraph 38, wherein the reference control tumor cell line is a highly metastatic tumor cell.
41. The kit of paragraph 38, wherein the reference control tumor cell line is a non-metastatic tumor cell line.
42. The kit of paragraph 38, further comprising instructions for assembly of the three-dimensional in vitro assay of paragraph 30.
43. The kit of paragraph 38, further comprising culture media.
44. The kit of paragraph 38, wherein the receiving substrate comprises extracellular matrix (ECM) cells in the hydrogel region.
45. The kit of paragraph 38, wherein extracellular matrix (ECM) cells are selected from the group consisting of: endothelial cells, stromal cells, fibroblast cells, or any combination thereof
46. A system comprising:
   a determination module configured to receive a separated substrates from a three-dimensional invasion assay to measure the invasive index of a cancer cell, and configured to output the amount of cancer cells in each substrate of the three-dimensional cellular assay after a predetermined period of incubation;
   a storage device configured to store the amount of cancer cells on each substrate three-dimensional cellular assay from the determination module;
   a comparison module adapted to receive input from the storage device and compare the data stored on the storage device with at least one reference invasive index; and
   an output module for displaying the information to the user.
47. The system of paragraph 46, further comprising an assay module.
48. The system of paragraph 46, wherein if the invasive index of the cancer cell is higher than a reference invasive index (e.g., for a non-malignant cell), the comparison module provides information to an output module that the cancer cell has high metastatic or invasive potential.
49. A composition comprising:
   c. a porous, hydrophilic seed-substrate comprising a plurality of defined regions comprising hydrogels disposed within a seed-substrate, wherein at least one hydrogel comprises a cancer cell population, and at least one hydrogel comprises a reference control cancer cell line; and
   d. at least one porous, hydrophilic receiving-substrate comprising a plurality of defined regions comprising hydrogels; wherein the receiving substrate is stacked above or below the seed-substrate.
50. The composition of paragraph 49, wherein the reference control cancer cell line is a positive control cell line comprising a highly-metastatic cancer cell line.
51. The composition of paragraph 49, wherein the reference control cancer cell line is a positive control cell line comprising a low-metastatic cancer cell line.
52. The composition of paragraph 49, wherein the reference control cancer cell line comprises a non-metastatic cancer cell line.
53. The composition of paragraph 48, wherein the reference control cancer cell line is a negative control cell line
54. The composition of paragraph 48, wherein the hydrogels in the at least one receiving substrate can comprise additional cells.
55. The composition of paragraph 53, wherein the additional cells are endothelial cells, stromal cells, fibroblast cells, or any combination thereof.

Having generally described this invention, the same will become more readily understood by reference to the following specific examples which are included herein for purposes of illustration only and are not intended to be limiting unless otherwise specified.

### EXAMPLES

The examples presented herein generally relate to a three-dimensional in vitro invasion assay and methods of use in assessing the metastatic potential of a cancer cell population. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods

Cell Lines.

The following invasive potential of the cell lines were assessed using the 3D in vitro assay as disclosed herein: Breast cancer cells; triple negative highly invasive cell line MDA-MB-231. Prostate cancer cells: highly invasive PC-3 and DU-154 isolate from bone and brain metastases, weakly invasive LNCaP and CWR-22 lines. Control cells included normal prostate epithelial cells (RWPE-1) and fibroblasts (HIN 3T3) as negative controls.

Culture of Cells in Multi-Layers Substrates.

Permeation of the suspension of cells in Matrigel into paper yielded thin (200 μm) layers of Matrigel with cells trapped within [Derda et al, PNAS 2009; Derda et al, PLoS submitted]. After 24-72 hours of culture, the cell-containing layer (“sender” or “seed-substrate”) was overlaid with one or more “blank” layers (e.g., “receiving substrates”) which comprise a paper matrix permeated with Matrigel free of cells. In some assays, one seed-substrate layer was sandwiched between two receiver-substrate layers (an upper-receiving substrate and a lower-receiving substrate). The inventors also assessed other combinations of different numbers of seed-substrates and different numbers of receiving-substrates.

After additional 1-5 days of culture of the 3-D assay of stacked layers, the layers were separated by peeling apart and the amount of cells (e.g., the number, density or quantity) of cells in one or more receiving layer was measured, and optionally the amount of the amount of cells (e.g., the number, density or quantity) of cells in the seed-substrate layer. In some instances, the number of cells that cross from the cell-containing sender layer to receiver layer.

To measure the quantity of cells in each layer, the layers were incubated in solution of calcein which stains viable cells and the layers were imaged using fluorescent scanner at 100 micron resolution.

### Example 1

The cells in gels in paper (CiGiP) was used and modified to develop an assay that measures the invasive capacity of multiple cell lines and primary cancer cells isolated from tumor biopsies. Stacking cell-containing layers permits simple control over 3D location of cells and multiples cell types. When the layers are separated after a pre-defined period of time allows for the assessment of migration of cells between the adjacent layers.

The inventors measured invasion of a panel of breast and prostate cancer cell lines with a known invasive capacity. The inventors demonstrate that the ability of cells cultured in seed-substrate layers permeated by a growth matrix, e.g., Matrigel to invade the adjacent Matrigel-containing layers of paper correlated with their invasive capacity in other in vitro assays.

These 3D CiGiP invasion assays are useful to identify the invasive capacity of cancer cells isolated from tumor biopsies. Additionally, the assays as disclosed herein are useful as an additional validation for the invasive capacity of the tumors and improve the quality of the cancer treatment.

The inventors demonstrate the simple procedures that can be performed by unskilled personal or robotics of seeding a biological sample comprising the cells to be tested on the seed-substrate layer, stacking a seed-substrate layer (comprising the test cells) on one or more receiving substrate layers, de-stacking the layers and measuring the quantity of cells on each substrate layer, e.g., by image analysis (e.g., scanning) or other quantitative measure such as PCR etc. As such, the inventors have demonstrated a simple multi-layered assay based on CiGiP which is suitable for high-throughput and high-capacity processing to assess the migratory capacity of cancer cells in 3D environment in vitro.

The inventors have also demonstrated that the assays are a simple and better model for pharmaceutical development than existing 3D invasion assay.

Using a multi-layer 3D culture assay as disclosed herein, in which there exist unidirectional gradients of oxygen and nutrients, the inventors demonstrated that cell migration occurs towards higher concentration of oxygen and nutrients (see FIG. 1).

The inventors also demonstrated that cells which are invasive (PC-3 and DU-154), invade the upper “receiver-substrate” layer (FIG. 1). This invasion is blocked by specific inhibitors of actin-based motility (cytoclasin D) confirming that invasion is a motility-based process. The number of non-invasive cells (LNCaP and CWR-22) that invaded the “receiver” layers is significantly lower than that of invasive cells, demonstrating accidental migration rather than invasive potential. One of ordinary skill in the art can optimize the assay by selecting layers with a predefined average pore size, or a uniform pore size or a substrate for the layer which is selected to prevent non-invasive cells from moving through the receiver layers unintended.

### Example 2

Cells were isolated from a core biopsy of a triple-negative breast tumor from human patients and cultured in paper permeated by Matrigel. After five days of culture, 50% of the cells invaded adjacent layers of paper that contained cell-free Matrigel. These data indicate that 3D CiGiP invasion assays can be used to diagnose the invasive capacity of cells isolated from tumor biopsies. These assays can provide additional validation for the invasive capacity of the tumors and improve the quality of the cancer treatment.

Plating the cells, stacking and de-stacking layers and scanning the samples are simple procedures that can be performed by unskilled personnel or robotics. Multi-layer assays based on CiGiP are suitable for high-throughput assays that assess migratory capacity of cells in 3D environment in vitro. Such assays can yield a simpler and better model for pharmaceutical development than existing 3D invasion assays.

Invasion of cells in vivo occurs in the presence of other cells, which are located in specific locations in three-dimensional extracellular matrix (ECM) around the migrating cells. Presence of other cells influences migration through cell-cell contact or through factors secreted by other cells and distributed in 3D environment via diffusion.

In some embodiments, it may be desired to use a more physiologically relevant invasion assay that includes the ability to position two or more cell types (e.g., cancer and stroma cells) in defined positions inside a physiologically-relevant 3D ECM matrix. For example, to mimic invasion of stroma, layers containing tumor cells and fibroblast cells can be overlaid, since the ability of cells to invade paper-supported stroma can differ from invasion of “blank” ECM.

To investigate the clinical relevance of CiGiP invasion assays, multiple samples isolated from patients with prostate cancer or samples from mastectomy can be profiled.

Experimental Design

Cells:

Breast cancer cells: triple negative highly invasive cell line MDA-MB-231. Prostate cancer cells: highly invasive PC-3 and DU-154 isolate from bone and brain metastases, weakly invasive LNCaP and CWR-22 lines. Normal prostate epithelial cells (RWPE-1) and fibroblasts (H₁N 3T3) were used as negative controls.

Cells were cultured in multi-layers substrates. Permeation of the suspension of cells in Matrigel into paper yielded thin (200 μm) layers of Matrigel with cells trapped within (data not shown). After 24-72 hours of culture, the cell-containing layer (“sender”) was overlaid with “blank” layers made by permeating paper with Matrigel free of cells (“receiver”). In most assays, one cell-containing sender layer was sandwiched between two receiver layers, but other numbers of cell-containing and blank layers were tested as well.

After an additional 1-5 days of culture of stacked layers, the layers were peeled apart and the number of cells in the sender layers was quantified. In addition, the number of cells that crossed from the cell-containing sender layer to the receiver layer was also quantified. For quantification, each of the layers was incubated in a solution of calcein, which stains viable cells, and the layers were scanned using fluorescent scanner at 100 micron resolution.

Results:

A multi-layered culture was assembled in which there exist unidirectional gradients of oxygen and nutrients, and the inventors demonstrated that the migration of cells occurs towards higher concentration of oxygen and nutrients. Cells which are invasive (PC-3 and DU-154), invade the top “receiver” layer. The invasion is blocked by specific inhibitors of actin-based motility (cytoclasin D) confirming that invasion is a motility-based process. The number of non-invasive cells (LNCaP and CWR-22) that invaded the “receiver” layers is significantly lower than that of invasive cells.

### REFERENCES

The references cited herein and throughout the application are incorporated herein by reference.

1. A method for assessing the metastatic potential of a population of cancer cells, the method comprising:
a. contacting a biological sample comprising a population of cancer cells at a defined region of a seed-substrate comprising a hydrogel, wherein the seed-substrate is a porous, hydrophilic substrate layer of a three-dimensional cellular assay, wherein the seed-substrate is in contact with at least one receiving-substrate to form a multi-layered three-dimensional cellular assay, wherein the receiving-substrate is a porous, hydrophilic substrate comprising a hydrogel at defined regions;
b. separating the seed-substrate and the at least one receiving-substrate of the three-dimensional cellular assay after incubation of the three-dimensional cellular assay for a predefined period of time; and
c. measuring the quantity of cancer cells on the at least one receiving-substrate, where the quantity of cancer cells on the receiving-substrate is indicative of the metastatic potential of the population of cancer cells.

2-48. (canceled)

49. The method of claim 1, wherein the seed-substrate is in contact with at least one lower-receiving substrate or at least one upper-receiving substrate, or at least one upper-receiving substrate and at least one lower substrate.

50. The method of claim 1, wherein the three-dimensional cellular assay comprises between 1 and 50 receiving substrates, or between 50 and 100 receiving substrates, wherein each receiving substrate is layered on at least one other receiving substrate.

51. The method of claim 1, wherein the at least one receiving substrate comprises extracellular matrix (ECM) cells or an additional cell type in the hydrogel.

52. The method of claim 1, wherein the seed-substrate and the at least one receiving-substrate comprises a matrix with a pore-diameter to allow invasion of metastatic cells.

53. The method of claim 1, wherein the porous, hydrophilic substrate of the seed-substrate and the at least one receiving-substrate comprises paper or a ceramic substrate with a pre-defined pore size.

54. The method of claim 1, further comprising measuring the quantity of cancer cells on the seed-substrate, wherein a high percentage of cancer cells on the seed-substrate as compared to the percentage of cells on the receiving substrate is indicative of a low metastatic potential of the population of cancer cells.

55. The method of claim 54, further comprising comparing the measured quantity of cancer cells on the seed-substrate with the measured quantity of cancer cells on at least one receiving-substrate at a predefined timepoint.

56. The method of claim 1, wherein the biological sample comprises a predefined number of cancer or tumor cells.

57. The method of claim 56, further comprising comparing the predefined number of cancer cells in a biological sample with the measured quantity of cancer cells on the receiving substrate at a predefined timepoint or the seed-substrate at a pre-defined timepoint

58. The method of claim 1, wherein the biological sample comprises at least one selected from the group consisting of; a portion of a cancer biopsy sample, or cell suspension of cancer cells or cancer cells obtained from a tumor biopsy obtained from a subject.

59. The method of claim 1, further comprising labeling the cancer cells, wherein the labeling of the cancer cells is performed when the cells are in the biological sample prior to contacting with the seed-substrate, or wherein the labeling of the cancer cells occurs within the three-dimensional cellular assay prior to, or after separation of the seed-substrate with the at least one receiving substrate.

60. The method of claim 1, further comprising fixing the cancer cells in the three-dimensional cellular assay prior to, or after separation of the seed-substrate with the at least one receiving substrate.

61. The method of claim 1, wherein the measuring the quantity of the cancer cells can be by any one or a combination of the following; image analysis, measuring gene expression or protein expression of predetermined selected genes, cell proliferation assays, fluorescent labeling assays, standard cytometry assays, heamocytometer, automated cell counter, and flow cytometer, wherein the cancer cells can be measured when present on the seed-substrate or at least one receiving substrate, or when they are removed from the seed-substrate or the at least one receiving substrate.

62. The method of claim 51, wherein the additional cells are selected from the group of endothelial cells, stromal cells, fibroblast cells, or any combination thereof.

63. A composition comprising:
a. a porous, hydrophilic seed-substrate comprising a plurality of defined regions comprising hydrogels disposed within a seed-substrate, wherein at least one hydrogel comprises a cancer cell population, and at least one hydrogel comprises a reference control cancer cell line; and
b. at least one porous, hydrophilic receiving-substrate comprising a plurality of defined regions comprising hydrogels; wherein the receiving substrate is stacked above or below the seed-substrate.

64. The composition of claim 63, wherein the reference control cancer cell line is selected from the group consisting of: a positive control cell line comprising a highly-metastatic cancer cell line, a positive control cell line comprising a low-metastatic cancer cell line, a non-metastatic cancer cell line, or a negative control cell line.

65. The composition of claim 63, wherein the hydrogels in the at least one receiving substrate can comprise additional cells or extracellular matrix (ECM) cells.

66. The composition of claim 65, wherein the additional cells are endothelial cells, stromal cells, fibroblast cells, or any combination thereof.

67. A method to identify if a subject has a metastatic cancer, the method comprising:
a. contacting a biological sample comprising a population of cancer cells obtained from the subject at a defined region of a seed-substrate comprising a hydrogel, wherein the seed-substrate is a porous, hydrophilic substrate layer of a three-dimensional cellular assay, wherein the seed-substrate is in contact with at least one receiving-substrate to form a multi-layered three-dimensional cellular assay, wherein the receiving-substrate is a porous, hydrophilic substrate comprising a hydrogel at defined regions,
b. separating the seed-substrate and the at least one receiving-substrate of the three-dimensional cellular assay after incubation of the three-dimensional cellular assay for a predefined period of time; and
c. measuring the quantity of cancer cells on the at least one receiving-substrate, where the amount of cancer cells on the receiving-substrate is indicative of the subject having a metastatic cancer.

68. A kit comprising:
a. at least one porous, hydrophilic seed-substrate comprising a plurality of defined regions comprising hydrogels disposed within;
b. at least one porous, hydrophilic receiving-substrate comprising a plurality of defined regions comprising hydrogels disposed within; and
c. a reference control tumor cell line.

69. The kit of claim 68, wherein the reference control tumor cell line is disposed within at least one hydrogel location of the seed-substrate, wherein the reference control tumor cell line is selected from the group consisting of: a positive control cell line comprising a highly-metastatic cancer cell line, a positive control cell line comprising a low-metastatic cancer cell line, a non-metastatic cancer cell line, or a negative control cell line.

70. The kit of claim 68, wherein at least one receiving substrate comprises extracellular matrix (ECM) cells in the hydrogel region.

71. The kit of claim 70, wherein the extracellular matrix (ECM) cells are selected from the group consisting of: endothelial cells, stromal cells, fibroblast cells, or any combination thereof.
